(19) United States
(12) Patent Application Publication
(10) Pub. No.: US 2021/0308333 A1
(43) Pub. Date: Oct. 7, 2021

(54) COMPOUNDS AND DEVICES HAVING TOPOGRAPHICAL COMPLEX SURFACE FOR WOUND HEALING

(71) Applicants: John E. Davies, (US); UNIVERSITY HEALTH NETWORK, Toronto (CA)

(72) Inventors: Niloufar Khosravi, Toronto (CA); Azusa Maeda, Toronto (CA); Ralph S. Da Costa, Etobicoke (CA); John E. Davies, Toronto (CA)

(21) Appl. No.: 17/251,525

(22) PCT Filed: Jun. 17, 2019
(86) PCT No.: PCT/CA2019/050853

§ 371 Date: Dec. 11, 2020

Related U.S. Application Data
(60) Provisional application No. 62/686,067, filed on Jun. 17, 2018.

(51) Int. Cl. A61L 27/50; A61K 49/00

(52) U.S. Cl. CPC A61L 27/50; A61K 49/0017; A61L 2300/442; A61L 2400/18; A61L 2430/02; A61L 2400/12

(57) ABSTRACT

Compositions, products and devices are provided for promoting wound healing. The compositions, products and devices have a topographical complex surface.

### FIELD

The disclosure relates to compositions and uses of a material with a complex surface.

### BACKGROUND

Each year, millions of metallic surgical implants are placed in patients worldwide, which include total hip replacements, dental implants and knee prostheses, screws to secure spinal fixation devices and anchorage components for facial prostheses, hearing aids and orthodontic appliances. With the exception of cemented prostheses, osseointegration is crucial to the functional success of such endosseous devices. Osseointegration may be achieved by either contact or distance osteogenesis—the formation of bone directly on the implant surface, or the old bone surface, respectively. While initial implant stability may be achieved by physical engagement in cortical bone, contact osteogenesis will only occur through bone remodeling. On the contrary, in the trabecular bony compartment, contact osteogenesis can provide rapid bony anchorage due to the recruitment and migration of osteogenic cells (osteoconduction) from the marrow interstices to the implant surface¹.

Nanosurfaces have improved clinical osseointegration by increasing bone/implant contact. Neovascularization is considered an essential prerequisite to osteogenesis, but no previous reports have examined the effect of surface topography on the spatiotemporal pattern of neovascularization during peri-implant healing.

As well, improved compositions and devices are needed that promote neovascularization during wound healing.

### SUMMARY

Compositions, devices, methods, and uses are provided that promote neovascularization during wound healing, and are preferably based on improved osseointegration seen in some bone implants.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 shows the Cranial Implant Window Chamber (CIWC) to study peri-implant vascularization and osteogenesis intravitally and longitudinally. (a) The cruciate Ti implant (4 mm in diameter) provided 4 distinct healing volumes that were selected as ROIs to examine microvascular growth and bone healing dynamics. [Note the central hole was designed to aid implant production and handling, as described in the text.] (b) Photograph of the CIWC surgically implanted in the calvaria of a living mouse. The cover-slip window was fixed peripherally with dental restorative material (white), stabilizing the implant in the defect. (c, d) A schematic demonstrating a mouse on the heated microscope stage. The head was immobilized by a custom-made metallic restrainer above and modeling clay below to minimize motion artifacts during intravital optical imaging. (e) The experimental timeline from the day of the surgery showing the subsequent imaging time points.

FIG. 2 shows imaging of the topography of the unmodified and modified surfaces by FE-SEM. Photomicrographs of the Ti implant surfaces at 20k, and 50k magnifications. (a and b) MA surface. (c and d) Grit blasted/Acid etched and nanotube (NT) surface. Scale bar (a and c) 2 μm (b and d) 1 μm.

FIG. 3 shows XPS of the implant surfaces. Survey spectra of (a) the MA and (b) NT surfaces. The two spectra appeared very similar in elemental composition indicating no significant surface contamination due to the multiple processing steps required for the NT surface. (c, d) Deconvolution of the Ti envelop for (c) MA surface and (d) NT surface. MA surface had a thin (<10 nm) TiO₂ layer as the underlying metal peak is seen at 453.2 eV (arrow). On the contrary, this and the sub-oxide peaks at <456 eV were absent in the NT surface, on which the oxide layer was >10 nm (the sampling depth of the technique).

FIG. 4 shows μCT images showing pattern of osteogenesis in response to implant surface topography. Images of the entire calvarial wound site including the Ti implant in the defect with the (a) MA and (b) NT implants at day 42 post-surgery. Note that the margins of the osteotomy are more easily seen with MA implants due to the fact that less bone has grown into the healing volumes. With NT implant, in one healing volume the surface of the implant is completely occupied with bone (arrow) and in another, bone is growing into the healing volume along the implant surface forming a Baud curve (arrowhead) typical of contact osteogenesis (see text). (c-f) Magnified images of the scans in (a and b) but at 2 different depths in the healing volumes around the (c, d) MA surface and the (e, f) NT surface. (d, f) represent the boxes marked in (A and B) but in each case, the same healing volume was imaged in a different plane in (c, e respectively) to provide additional information. Again, a Baud curve of contact osteogenesis was clearly seen in (e-arrowhead) but was absent in the MA sample. Scale bars (a, b): 1 mm. (g) Quantitative analysis of bone regeneration parameters (Bone Volume/Total Volume and Bone Implant Contact) in MA and NT groups at day 42 post-surgery. Data is shown as mean±SD (n=4, ***=p<0.001) (h) Schematic showing the coronal view of the CIWC.

FIG. 5 shows in vivo longitudinal microvascular response to MA and NT cranial implants. Representative overlaid images of two channels: Silver gray—the reflected light (622-666 nm) Ti implant; Green—the FITC-DEX labeled vessels at day 7 post-implantation, showing little neovascularization around the (a) MA implant compared to (b) NT implant. Notes: (1) the concentric lines seen clearly on MA surface are machining marks and are less obvious on the NT sample; (2) The leakage of FITC-DEX from the tip of the incompetent, newly forming, vessels was obvious at higher magnification (b.1 and b); (b.2) Green and silver grey channel overlaid (B.b) Green channel (c) Representative images demonstrating formation and development of the peri-implant neovascular network from day 3 to day 42 around the MA surface and the NT surfaced implants. (e) The comparative quantification of functional vessel density (see text) between implant groups over time. N=8 mice/group/time-point. Results are mean±SEM. *p-value<0.05, ***p-value<0.001. Scale bars (a-d): 500 μm. (b. 1 and 2): 200 μm. Images are stacks of tiled scans of the entire craniotomy at the maximum intensity projection; the depth of the field is 0.5 mm, which is equal to the thickness of the implant.

FIG. 6 shows vascular progression, regression, and remodeling on the NT over time. Silver gray—the reflected light Ti implant; Green—FITC-DEX blood vessels at day (a) 11 (b) 15, and (c) 22 post-surgery. 2 independent locations (rows L1 and L2) on the NT implant surface are shown for each time point. (a) Neovessels formed and partially anastomosed around the Ti implant by day 11. (b) Formation of vascular loops with functional circulation (arrowheads) by day 15. (c) Vasculature remodels to produce fewer but more mature, and larger, functional vessels by day 22. hv=healing volume; ch=central hole. Scale bar: 500 μm.

FIG. 7 shows changes in vascular network structure and branching statistics in response to implant surface topography at day 7 post-surgery. (a, b) Images of the vascular network proximal to the MA and NT surfaces. Green—blood vessels; Silver gray—Ti implant. Each healing volume can be described by 3 Cartesian coordinates. The X and Y axes are marked (the z axis would be the depth of the healing volume). (c, d) Corresponding 3D image skeletons showing the spatial distribution of the vessel segments in two representative HVs. The coordinates were anchored by defining the bottom left corner of the HV as the point of reference (0), and the implant was located at the top right of the HV. The color coding represents the vessel branching number. (e, f) Box plots represent the quartile distribution of the vessel segment coordinates along the X and Y axis in MA (Black) and NT (Green) groups, with the whiskers representing the 0 reference point and the surface of the implant respectively on the X and Y axes. The individual data points are superimposed along the whiskers as scatter plots. The higher the value X or Y, the closer the segments were to the lateral surface of the implant. (g) Comparison of the branching number between MA and NT groups. N=8 animals/group/time-point. Error bar=median±IQR. Scale bar is 200 um.

FIG. 8 shows comparison of the vascular morphometric parameters between implant types characterizing the features of the vessel network: branching number; functional vascular volume, and vessel length from day 7 to day 15 post-surgery. In all: Black=MA day 7; Gold=MA day 15; Green=NT day 7; Red=NT day 15. Branching number of the network in (a) MA and (b) NT groups. (c) Functional vessel volume in NT was significantly higher than those of the MA implants in both time points; error bar=Mean±SEM. (d) There was a significant difference between the vessel lengths in the MA and NT groups at both time points. Error bar=median±IQR. Note: significant difference in all parameters between time points observed in NT groups indicates that the functional attributes of the vascular plexus change to improve the blood flow in the wound site from day 7 to day 15. No significant difference in branching number and vessel length was observed between time points in MA implant due to lack of hierarchical branching and anastomosis; however, the functional vascular volume increased significantly. N=8 animals/group/time point. NS=Not Significant, **P-value<0.01, ***P-value<0.001.

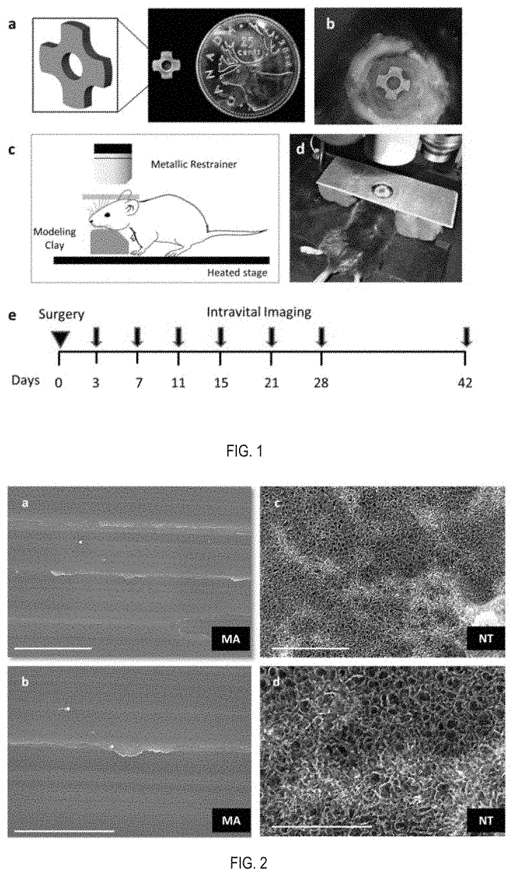

Supplementary FIG. 1 shows μCT images of the healing volume around the NT Ti implant over time. No bone has been formed in the healing volume around the Ti-implant at (A) 2 weeks post-implantation (B) at 4 weeks the bone formation is evident on the surface of the implant and in the healing volume (arrowheads).

Supplementary Movie Still 1 shows intravital live video of peri-implant neovasculature. Peripheral and central vasculature anastomosed on the top flat surface of the implant, facilitating the blood flow in each direction.

Supplementary Movie Still 2 shows μCT images of the entire healing volume around the NT Ti implant at day 42 post-surgery. The stack of the images along the Z axis shows different slices from the top (cover glass as seen in the ground glass appearance due to shadowing) through the depth of the healing volume. Formation of the bone on the surface of the implant is apparent. As the endocranial periosteum (dura mater) is approached, we see no signs of bone formation originating from the dural surface.

FIG. T1. In vivo longitudinal imaging of the cellular phenomena during the peri-implant wound healing using Hic1CreERT2 reporter mouse model. (a) Cross-breeding Hic1CreERT2 with RosaLSL-tdTomato mice generated offspring expressing tdTomato positive mesenchymal progenitor cells (MPs). To induce CRE-ERT2 nuclear translocation, 8 weeks-old mice were administered by Tamoxifen. A 10-day washout period was allowed before CWIC placement surgery followed by longitudinal imaging. (b) Top view of the CIWC in mouse skull after surgery. (c) Implant surfaces TiNT and TiMA was taken by scanning electron microscopy. Scale bar 500 nm. (d, e) Full view of the TiNt and TiMA implants and the regeneration process images using confocal microscope at day 7 post-surgery. Blood vessels (RTC), MPs (tdTomato), Titanium implant visualized by collection of the reflected light. (f) Longitudinal intravital imaging of the MPs recruitment to the site of the implant at various time point from day 3 to 43 post-implantation at both TiNT and TiMA. (g) Quantification of the number of the cells over time at both implant groups. N=8 mice/group/time-point. Results are mean±SEM. Two-way ANOVA was performed followed by Bonferroni post-test to compare the mean differences between implant groups over time and at each time point individually ***p=value<0.001. Scale bar (d-f)=500 μm.

FIG. T2. Nanosurface affects the population of the progenitor cells in the peri-implant wound site which also correlates with neovascularization. (a) Longitudinal imaging of the FITC-Dex infused blood vessels (green) and tdTomato Hic1⁺ MPs (red) in a healing volume around the TiMA and TiNT implants from day 3 to 28 post-implantation. (b) Shows an example of the geometry of the healing volume which is viewed in each of the images in (a), Silver gray—the reflected light (622-666 nm) Ti implant. The dotted line marks the bone periphery, HV=healing volume. In (a) at TINT group, a substantial increase in the number of the cells (2.9 fold) was observed at day 7 post-surgery which continued till day 11 but started to diminish at day 15 and 28. A bloom of tdTomato cells was apparent in the periphery of the defect. In the TiMA group, the number of tdTomato cells gradually increased from day 3 and day 15 and went down from day 15 to day 28. The bloom of tdTomato cells is absent from the periphery of the defect bearing the TiMA implant. (c,d) The quantification of functional vessel density and the number of the MPs in a healing volume in early time points (day 3 to day 11) at both implant groups shows that the growth of the new vessels and the population of the wound site by MPs happens simultaneously. The Pearson correlation coefficient r close to 1 shows a positive correlation between the two parameters, N=8 mice/group/time-point. Results are mean±SEM. Scale bar (b)=500 um.

FIG. T3. The majority of the Hic1⁺ cells are located in the inner layer of periosteum and follow the vascular growth pattern. Representative intravital image of a healing volume around a TiNT implant at day 3 (a-c) post-implantation. Green—Blood vessels, Red—MPs, Silver grey—Titanium implant. (a) shows the maximum intensity projection of the 3D stack of images with all 3 channels. 3D stack of images along the Z axis is splited into two compartments: (b) the body of the wound and thus closer to the glass cover-slip (Z=100-200 μm) and (c) the deeper compartment close to the dura mater (Z=0-100 μm). Green and Red channels are shown in b and c. Note: in the body of the wound, Perivascular cells are not bound to the newly growing vessels (b). However, the undamaged blood vessels in the dural tissue are covered with pericytes. (d) Representative intravital image of a healing volume around a TiNT implant at day 7 post-implantation. (e) A color-coded map of the 3D distribution of the tdTomato MPs in the healing volumes rendered in Imaris software (color scale: 0.025 to 0.275 μm distance along the Z-axis perpendicular to the skull). (f and g) The cross-sectional views clearly show that the majority of the MPs cells were in the upper half of the entire optical section which is close to the periosteum. Note: the migration pattern of MPs from the periphery of the defect towards the surface of the implant in the coronal view of the healing volume. (h) FITC channel for blood vessels overlaid on the map tdTomato cells. (f) Histological section of mouse cranium, H&E staining, shows the different anatomical zones within the cranium. Cortical bone (marked c) diploe (marked d), the top layer above cortical bone is periosteum and the outer layer of the endocranial side is duramater. Scale bar (a-c)=200 μm, (d,e,g,h)=150 μm, (f)=100 μm.

FIG. T4—Microanatomical location of the tdTomato⁺ cells in bone and their contribution in defect healing. (a) RFP expression was detected by immunohistochemistry in the periosteum, diploe, and duramater at day 42 post-implantation (black arrowheads). The majority of the RFP expressing cells were in the inner layer of periosteum. (b-g) Consecutive sections stained for H&E, RFP, and CD31 in both intact and injured craniums in 12-week-old mice. (h) Quantification of RFP positive area (% of the total image) shows a 3-fold increase in the number of MPs in the defect model compared to the non-defected controls. (i) The expression of CD31 showed a nonsignificant increase in the vascularization within the tissue defect compared to the intact cranium. T-test has been performed to test the significance of the results, ***P-value<0.001, *P-value<0.05.

FIG. T5. tdTomato is expressed in at least 3 phenotypically different cell populations within the peri-implant niche. Visualization of tdTomato cells in calvarial defect at different timepoints post craniotomy. High magnification IVM (a) tdTomato cells are in the proximity of newly forming blood vessel. No pericytic coverage is seen on the new leaky blood vessel. (b) more blood vessels are formed, pericytic coverage is increased. (c) A portion of tdtomato cells have become pericytic and stabilized on the new vessels. (d-f) At day 15 post-implantation, various morphologically distinct cells are visible in the wound niche expressing tdTomato. Green is FITC-Dex blood vessels, red is tdTomato Hic1⁺ cells. “P”, a pericyte like cell closely juxtaposed to the vessel wall and having cell processes that envelop the vessel. Fibroblast-like cells showing a migratory morphology “F1” long and spindle-shaped, migrating within the 3-dimensional matrix, or “F2” flattened with a leading edge and trailing tail. (g) Addition of SHG channel allows visualization of bone and collagenous matrix at day 21 post-implantation. Blue arrowhead shows the fibrous tissue. (h) is the overplayed image of red and green channels, “O” is a tdTomato osteocyte with cell processes buried within the bone. Scale bar (a-h)=100 um, (i)=500 um.

FIG. T6. Flow cytometry analysis of mesenchymal stem cell marker expression in P3 Human Umbilical Cord Perivascular Cells. (a-f) Representative flow cytometry analysis of human umbilical cord perivascular cells stained for HLA-DR, CD90, CD45, CD10, CD31, CD73, CD105, CD166, CD146, CD140b, CD34, and MHC1. (g, h) Matching isotype controls. (i,j) Flow cytometry data are presented as a positive % expression or mean fluorescence intensity (MFI), which is a measure of the intensity of the signal. Values are mean±SEM. N=7 is the number of replicates.

FIG. T7. Characterization of Hic1 perivascular, and endothelial, cell locomotion in response to a gradient of platelet growth factors, (a) Chemotaxis μslide set-up. A narrow observation area connects two larger reservoirs. The cells (HUCPVCs and HUVECs) were initially seeded in the observation area, the left reservoir was filled with platelet lysate (PL) and the right reservoir was filled with culture medium. By diffusion, cells were exposed to a linear gradient of the PL. (b) View of the HUCPVCs seeded in the observation area taken by live-cell video microscopy for 48 hrs, visualized by phase contrast and DAPI. Scale bar 200 μm. (c) Expression of Hic1 in HUCPVCs in comparison with bone marrow mesenchymal cells (BM-MSCs)—a common source of MSCs—determined by microarray. (d) Trajectory plots showing the path of migrating HUCPVCs under the concentration gradient of platelet lysate (+/−), positive control (+/+), both reservoirs filled with Platelet lysate and negative control (−/−), the serum-free culture medium in both reservoirs (SFM/SFM). At least 41 cells have been tracked for each experimental condition for 3 technical replicates (N=3). In control experiments, cells are moving randomly and distributed uniformly around the origin. However, the migration of the cells is directed to one side under influence of the gradient. P-value of the Rayleigh test=1.9E-9, 0.06, and 0.1 respectively, confirms the significance of the directed migration under PL gradient. Forward Migration Index, X=direction parallel to the gradient of PL, and cell speed for (e) HUCPVCs (f) HUVECs. The number of migrating cells under the various concentrations of PL (g) HUCPVCs (h) HUVECs. The data represent means±SD (N>4). **P-Value<0.01, *P-value<0.05. (i) Time-lapse images of an endothelial cell migrating in platelet lysate gradient.

### DETAILED DESCRIPTION OF THE DISCLOSURE

It is generally accepted that the mesenchymal progenitors of osteogenic cells are perivascular cells^{2,3}, although little is known about how and when these cells enter the wound site. Neovascularization, or formation of new blood vessels, is a critical prelude to osteogenesis. Neovascularization may occur through either angiogenesis and/or vasculogenesis^{4,5}; and it can be assumed that the incursion of perivascular cells is dependent upon neovascularization. Neovascularization may occur through a variety of mechanisms⁶⁻¹¹ that lead, through maturation, to the establishment of a hierarchical functional vascular network. While implant surface design is considered a critical driver of osteoconduction, and topographically complex implants have been shown to increase bone-implant contact (BIC)¹²⁻¹⁴, no evidence has emerged to suggest that implant topography has an influence on peri-implant neovascularization.

It has been shown that implant surfaces increase platelet and neutrophil adhesion and activation¹⁵⁻¹⁷ that lead to an increased level of local angiogenic and osteogenic growth factors and cytokines¹⁸. Furthermore, micron-scale roughness on titanium (Ti) implants has been shown to stimulate the secretion of pro-inflammatory cytokines by macrophages including tumor necrosis factor (TNF)-α¹⁹, which primes endothelial cells for angiogenic sprouting²⁰. Indeed, some authors have reported that rough implant surfaces affect endothelial cell proliferation, motility²¹, and endothelialization (tube formation)²². To complement these in vitro reports, upregulation of angiogenic and osteogenic genes has been reported following clinical insertion of topographically complex titanium implants²³.

To determine the effect of surface topography on peri-implant healing, the inventors have developed a new in vivo experimental murine model to track the spatiotemporal development of neovascularization in the peri-implant healing compartment as a function of implant surface topography. The model integrates a custom-designed cranial metallic implant with an optically-transparent window chamber that is compatible with both confocal- and multiphoton-based intravital microscopic imaging systems.

From these models and studies, implant surfaces that promote osseointegration are utilized to develop materials that promote or enhance neovascularization for wound healing, such as subcutaneous or internal wounds.

In some embodiments, a composition for the promotion neovascularization during wound healing has a topographical complex surface, such as a micro- and/or nano-topographical complex surface. In one embodiment, the composition is made of a biocompatible material. Examples of biocompatible materials include, but not limited to: degradable synthetic and biological polymers, co-polymers, polymer blends, rubber, latex, silicone, carbon materials and inorganic materials such as metals, silicon, glass, ceramics and composite/alloy materials.

As used herein, a “topographical complex surface” means a surface structure in the micro or nano scale. In some embodiments, a topographical complex surface is comprised of microtubules, threading, pores, porous sinters, and/or microtextures. Examples of topographical complex surfaces are found in Koshy, E., and Philip, S. R. (2015); Smeets, R. et al (2016), and Stanford, C. M. (2010), the entire disclosures of which are incorporated herein by reference.

In some embodiments, the composition having micro- and nano-topographical complex surface can be formed into a product or device. In other embodiments, a product or device is provided comprising the composition having micro- and nano-topographical complex surface. Examples of such a product or device include, but are not limited to: skin dressing, bandage, scaffold, patch, implant, thin film, wire, catheter (insertion lines), meshes, nanowires, and implantable vascular beds.

Embodiments of the compositions can be of any size or shape or thickness and can be formed into in at least one of the product or device, or combinations thereof.

Compositions having a micro- and nano-topographical complex surface, and products or devised formed from such compositions can be used for modifying the rate, extent, location and directionality of vascularization (as well as cell migration and cytokine release) for tissue regeneration, cell therapy, organ transplantation, wound and defect healing, and cosmetic and agricultural engraftment applications. In one embodiment, the compositions, products or devices are used for modifying or enhancing the rate, extent, location and directionality of neovascularization during wound healing.

In some embodiments, the compositions, products, or devices are used in combination with biological components. In other embodiments, the compositions, products, or devices further comprise biological components. Examples of biological components include, but not limited to: tissues, cells, exosomes, extracellular vesicles, microparticles, cytokines, drugs, antibiotics, antifungal, anti-inflammatory, nanoparticles, and media.

In some embodiments, the compositions, products, or devices are used in combination with contrast agents. In other embodiments, the compositions, products, or devices further comprise contrast agents. Examples of contrast agents include, but are not limited to: fluorescent dyes, chromogenic dyes, quantum dots (QDots), Raman-active agents, molecular beacons, nanoparticles having fluorescent agents, and scattering or absorbing nanoparticles, biologically-activated/sensitive contrast agents (enzyme-cleavage, pH-sensitive, ROS-sensitive).

In some embodiments, contrast agents are used to label various components of the micro- and nano-topographical complex surface of the composition and any additional components, such as biological components. For example, the nanosurface could be optically labeled with a fluorescent dye of a specific fluorescence wavelength and impregnated cells could be labeled with another fluorescent dye of a different fluorescence wavelength, and each dye excited by different wavelength light sources. In this fluorescence multiplexed manner, different components of the embodiment can be labeled and tracked in a target over time to determine changes therein.

In tissue regeneration, cell therapy, organ transplantation, wound healing and cosmetic applications, the compositions, products, and devices are used to increase the loco-regional amount of functional blood vessels (as well as cell migration through and cytokine release from) a target tissue or organ or wound to improve the treatment thereof.

In some embodiments, the compositions, products, or devices are applied or inserted or administered or implanted in a target. As used herein, examples of a target includes, but are not limited to: a surgical field, a wound, a burn, a tumor, an organ, a tissue or cartilage or tendon, a scar target, a skin target, a biological target, a non-biological target, an oral target, an ear-nose-throat target, an ocular target, a genital target, a bladder target, a gastrointestinal target, a facial target, a cardiac target, a lung target, a bone and non-bone orthopedic target, a cartilage or spinal cord target, an anal target and a body target, a body defect target, a nerve target, a surgical cavity target, an engineered tissue construct, a plant material target.

In some embodiments, the products or devices described herein are used in ameliorating the adverse effects of aging or impaired healing conditions brought about by diseases impeding healthy functional vascularization e.g. diabetes, macular degeneration, or to increase or restore vascularization in skin grafts from autologous or substitute graft sources, increasing vascularity in damaged heart disease.

In one embodiment, the products or devices described herein employ one or more integrated or embedded contrast agents which can be interrogated using imaging or spectroscopic means to detect a change in the micro- and nano-topographical complex surface. This is useful for monitoring aspects of the composition when applied or inserted or administered or implanted in a target or patient. The use of embedded contrast agents could provide a means of monitoring the presence, decay, absorption into the target, efficacy of therapeutic effect size.

The following non-limiting examples are illustrative of the present disclosure:

### EXAMPLE 1

We have developed a cranial window model to study peri-implant healing intravitally over clinically relevant time scales as a function of implant topography. Quantitative intravital confocal imaging reveals that changing the topography (but not chemical composition) of an implant profoundly affects the pattern of peri-implant neovascularization. New vessels develop proximal to the implant and the vascular network matures sooner in the presence of an implant nanosurface. Accelerated angiogenesis can lead to earlier osseointegration through the delivery of osteogenic precursors to, and direct formation of bone on, the implant surface. This study not only highlights an important aspect of peri-implant healing, but also informs the biological rationale for the surface design of putative endosseous implant materials.

We used the model was to determine the outcomes of contact and distance osteogenesis on nanotopographically complex (NT) and machined-surfaced (MA) implants, respectively. Then, we demonstrate that differences in the topography of the surface are reflected in significantly different patterns of peri-implant neovascularization.

### Materials and Methods

### Animal Studies:

All animal procedures conducted in accordance to institutional animal use guidelines approved by University Health Network animal care committee (AUP #4884.0-1). Nine to eleven-week old male C57BL6 mice (Charles River Laboratories, Quebec) were used for the entire study.

### Titanium Cranial Implants:

The implants were custom-made from grade IV commercially pure titanium, specifically for this study, by ZimmerBiomet Dental, (Palm Beaches Garden, Fla.). The implants were machined from a 4 mm rod stock with a central 2 mm drill hole. Four radially equidistant flutes, with internal radii of 0.5 mm, were machined along the length of the rod. The rod was then machine-sliced, resulting in flat, 4 mm diameter and 500 μm thick, implant forms with the cruciate shape as seen in FIG. 1a. The topographies of all surfaces of such implants bore the marks of the machining process. The first cohort of the machined implants were left unmodified (MA) while the second cohort (NT) was further modified by bolting multiple implants together, through the central hole, and using guide bars to ensure that the flutes were aligned longitudinally. The outer surfaces were then grit blasted [325-450 um particle size range] dual acid etched in 8% hydrofluoric acid (HF) followed by 78% H₂SO₄/3% HCl (vol %), and TiO₂ nanotubes (NT) were created on this modified surface by electrochemical anodization. For this, the machined implants were ultrasonically cleaned in concentrated detergent followed by rinsing in deionized (DI) water. The NT implants were anodized in an electrolyte consisting of 0.250 wt % HF) (Sigma Aldrich™). The titanium implant served as the anode while a cp-titanium electrode served as the cathode. Both were connected to a power supply (BK Precision 9602) at 20V and immersed in the electrolyte solution with stirring at room temperature for 30 min. After anodization, the implant was rinsed with DI water and air dried at 120° C. for 1 h in a forced convection oven. The central bolt was removed and the modified individual implants were ultrasonically cleaned in acetone, 70% ethanol, and deionized water, and subsequently autoclaved at 121° C. for 20 min. A total of 10 MA and 10 NT implants were used in this study. The resulting implants, therefore, had a cruciate form of 4 mm external diameter, a central hole of 2 mm, and 4 cut-outs that provided 4 separate tissue healing volumes. Implants were individually packaged and sterilized by gamma-irradiation.

### Implant Surface Characterization:

Field emission scanning electron microscopy (FE-SEM): Two Ti implants from each surface group were removed from the sterile packs with plastic tweezers and fixed with carbon tape to SEM stubs, taking care to not to damage or contaminate the surfaces. Both the lateral and flat surfaces of the implants were imaged non-coated at an accelerating voltage of 5 keV and increasing magnifications (up to 50,000×) by FE-SEM (Hitachi S-5200, Japan).

X-ray photoelectron spectroscopy (XPS): Implants were analyzed by XPS using a Thermo Fisher Scientific K_{α} spectrometer (E. Grinstead UK). A monochromatic Al K_{α} X-ray source was used with a nominal 400 μm spot size. Survey spectra were obtained (200 eV pass energy (PE)) followed by an examination at 150 eV PE of spectral regions of interest from which the relative atomic percentage composition was obtained. High-resolution spectra (25 eV PE) were also obtained for the Ti envelope. Charge compensation was applied for all spectra using a combined e/Ar+ floodgun, and the energy scale was shifted to place the C1s peak at 284.6 eV. All data processing was performed using the Avantage 5.926 software supplied by the manufacturer.

### Surgical Procedure for Cranial Implant Window Chamber (CIWC) Placement:

The surgical procedures were performed in a microsurgery room under aseptic conditions on a microsurgical table. Mice were anesthetized with Isoflurane 2.5% vaporized in a 70/30 mixture of O₂/N₂O. The scalp was shaved and the skin was cleaned with Betadine solution and 70% ethanol. The skin was lifted with tweezers from the midpoint of the ears, cut with fine curved dissecting scissors, and completely removed to expose an 8 mm diameter circular area in the underlying skull. The periosteum was reflected using a periosteal elevator. Once the calvaria was exposed, a midline 4 mm diameter osteotomy was carefully created in the parietal bones using a custom-made trephine (ZimmerBiomet Dental, FL) under continuous irrigation with sterile saline. A guidance stop-line, laser-marked at 200 μm from the tip of the trephine, minimized over-penetration into the craniotomy site during the surgery. The created circular bone piece within the osteotomy was elevated using a periosteal elevator, taking care to leave the dura mater intact. An implant was then placed into the osteotomy. A permanent intracranial imaging window was superimposed over the implantation site to permit imaging through the depth of the healing volumes (and central implant hole), secure the implant, and inhibit the growth of the skin over the defect (FIG. 1b).

To fix the imaging window, the exposed skull around the osteotomy was first covered with Scotchbond dual cure adhesive resin and then a ring of dental restorative material (3M, Milton, ON) was applied on top of the bonding agent but maintaining a 3 mm distance from the edge of the craniotomy. A round coverslip, 8 mm in diameter, #1 thickness (neuVitro, Germany) was positioned on top of the restorative ring, pressing down gently to secure the Ti implant in the craniotomy. The restorative material was then light-cured to ensure a perfect seal around the defect and to stabilize the coverslip on top of the defect. Physiological body temperature was maintained throughout the surgery and recovery time by a homeothermic pad and healing lamp. Animals were carefully monitored after CIWC placement and they resumed normal activities within 3 days.

### Intravital Confocal Laser Scanning Microscopy:

Intravital imaging was performed post-operatively to track the microvascular changes during peri-implant healing. Prior to each imaging session, mice were anesthetized by standard intraperitoneal injection of a ketamine/xylazine mixture [80/13 mg/kg]. Each mouse was then administered FITC-DEX (2 MDa; 0.1 mg/mouse; 200 uL injected/mouse) via the tail vein using an ultra-fine 6 mm insulin syringe.

Creation of motion artifacts caused by respiration was controlled and minimized by stabilizing the mouse head on modeling clay and resting the body on a heated stage. In addition, the imaging window was fixed in place by fitting it into a metallic restrainer as demonstrated in FIGS. 1c and d. The imaging procedure was followed according to the experimental timeline shown in FIG. 1e. The confocal/two-photon fluorescence imaging was performed using LSM 710 (Carl Zeiss, Germany). Using the XYZ-axis controller, the crucial landmark locations, such as bone-implant interface, were identified through the CIWC. Once the implant interface and the adjacent bone were found, the images were acquired using 5×, 10×, and 20× water immersion objectives. Images were acquired with 488 nm excitation and 500-550 nm emission at 1024×1024 pixels and 0.79 μs pixel dwell. The implant was visualized by collecting reflected light in a second channel (633 nm excitation, 622-666 nm emission).

To obtain 3D images of the CIWC, the points above and below the implant in the z-plane were defined by driving the microscope to a point just out of focus on both the top and bottom of the implant surface. Images were recorded as a series of TIF files with dimensions of 1024×1024 pixels. Stacks of images were collected for the FITC channel with Z-stack size≅500 μm. Image acquisition settings were maintained consistent throughout all time points and groups.

### Image Processing and Analysis:

Fluorescent images were processed in Zen lite (Zeiss, Jena, Germany) and ImageJ. A MATLAB-based computational code was developed to calculate the functional vessel density. To calculate functional vessel density, maximum intensity projection images of the z-stacks were obtained, binarized and the positive pixel percentage area was calculated for each Region of Interest (ROI).

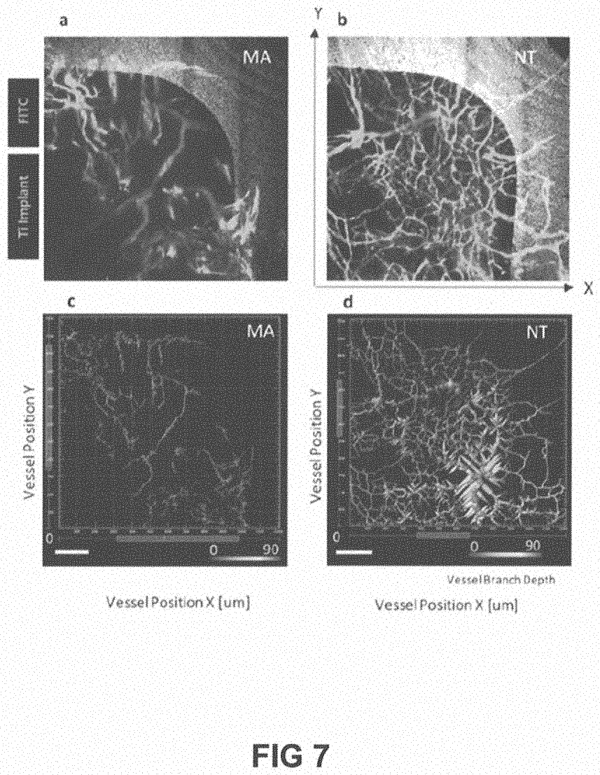

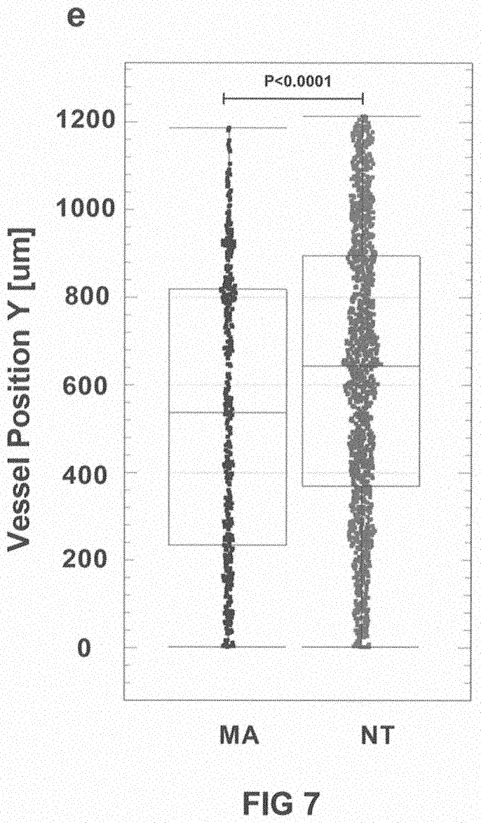

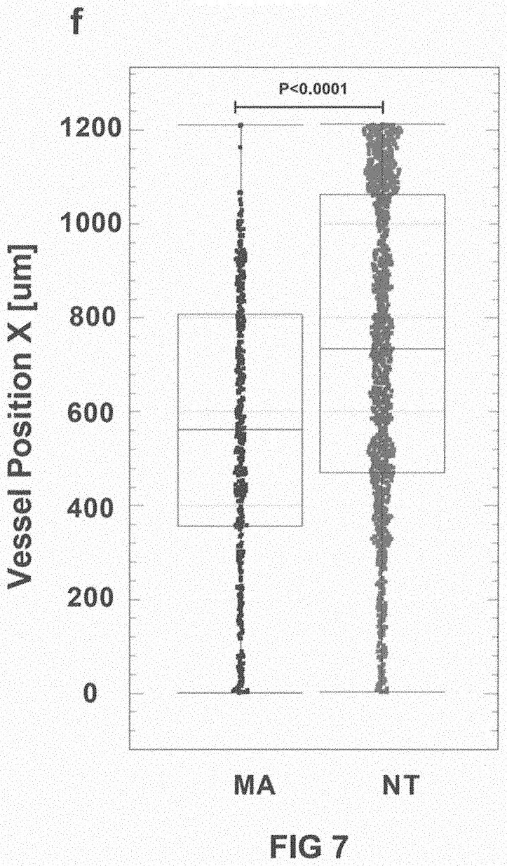

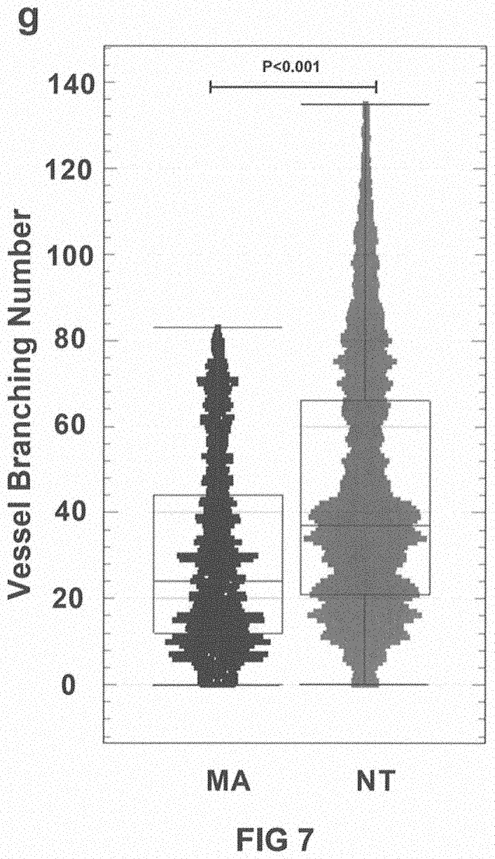

Quantitative spatial analysis of the vascular network structure in 3-D was performed using the Imaris (ver. 8.3.0, Bitplane AG, Switzerland). The 4 healing volumes represented 4 ROIs that we identified and analyzed at each imaging time point. To measure vessel parameters, each implant healing volume is first oriented in the same manner, as shown in FIGS. 7a and b Each healing volume can then be described by 3 Cartesian coordinates, the X and Y axes are marked in FIG. 7b (the z axis would be the depth of the healing volume, which is not seen in this plan projection). Stacks of images were analyzed using the filament tracer function. The size of each ROI was 1200×1200 micrometer², and all 4 ROIs were set at the same orientation to maintain the coordinates consistent across all images. A semi-automatic looping algorithm was used to detect and skeletonize the vascular network, The following definitions were employed: filament: the stem vessel including all branches; segment/branch; the distance between two branch points or between a branch point and a beginning/terminal point in a filament. Filaments are the building blocks of a vascular network. The following parameters were analyzed: vessel branching number; the number of branch points in the shortest path from the beginning point to a given point in a filament. Vessel position X and Y: the X and Y coordinates of a vessel segment positioned on an XY plane with respect to a reference point. Vessel volume: the sum of the volumes of all segments within the entire filament, and vessel length: the sum of the length of all segments within the entire filament.

### Sample Harvesting and Ex-Vivo Micro-CT Imaging:

The animals were euthanized by exposure to CO₂ at days 14, 28, or 43 post-surgery. The complete skull was harvested and fixed in 10% formalin for at least 48 hrs. Following fixation, the mandible and the brain were removed, and the dura was kept intact. The samples were further trimmed to remove excess tissue for μCT scanning.

Prepared trimmed samples were scanned using a MicroCT40 (Scanco Medical, Switzerland) at 70 kVp and 114 μA. Images were acquired with a high resolution in three planes, creating slices of 6 μm-thick. A ROI that included the entire defect area was selected, and highlighted in the cross-sectional images from each specimen. ROIs were then reconstructed in 2-D enabling visualization of bone formation in each of the 4 healing volumes in each implant. 2-D images were used as a qualitative demonstration of the mechanism of bone formation (contact vs. distance osteogenesis) at the healing volumes.

### Statistical Analysis

Temporal series results (Day 3 to 28) were presented as mean±SEM, and analyzed by two-way repeated measures analysis of variance (ANOVA) in Graphpad. Bonferroni post-tests were performed to test the significance of the means between implant groups at each time point. A confidence level of 95% was considered significant. The in vivo optical imaging procedure was repeated with 6 to 8 animals per time point per implant group. To obtain the statistics of the (vessel) filaments, a D'Agostino-Pearson normality test was performed to assess the normality of all data sets. As the data was not normally distributed, where two implant types were compared at one time-point, Mann-Whitney test was used to assess the statistical significance of the two medians; Interquartile range (IQR) has been shown on the scatter dot Mann-Whitney plots. Where comparing 3 or more groups of data, a Kruskal-Wallis test was performed followed by a Dunn's multiple comparison test. P-values<0.01 were considered significant.

### Results

### Cranial Implant Window Chamber (CIWC) Model

The CIWC was designed to fit precisely into a trephined calvarial defect of 4.0 mm diameter (FIG. 1a). The friction fit between the periphery of the implant and the marginal bone provides the initial stability of the implant. The four cut-outs, making the cruciate shape, provide four distinct healing volumes (regions of interest) to examine neovascularization and bone formation over time (FIG. 1b-e), which are two crucial steps to integrate the Ti implant in the calvarial bone. We employed nanotopographically complex (NT) and machined (MA) implants.

The surface topographies of both MA and NT implants were characterized by field emission scanning electron microscopy (FE-SEM). At lower magnifications machining marks were still visible on the MA implants (FIG. 2a), but at higher magnifications they were essentially devoid of surface features (FIG. 2b). On the contrary, NT surfaces showed both the micron-scale topography created by grit blasting and acid etching (FIG. 2c), and a superimposed nanotopography due to the creation of nanotubes (FIG. 2d).

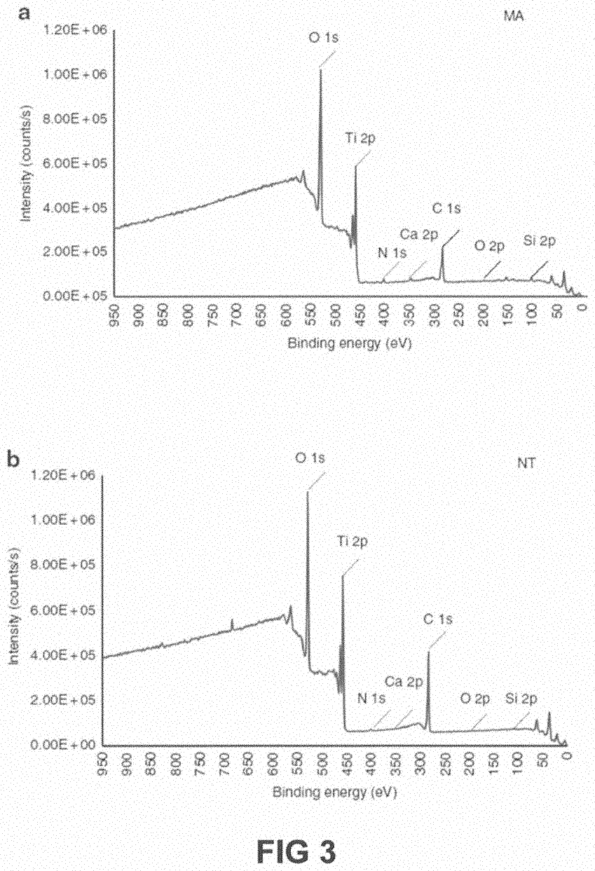

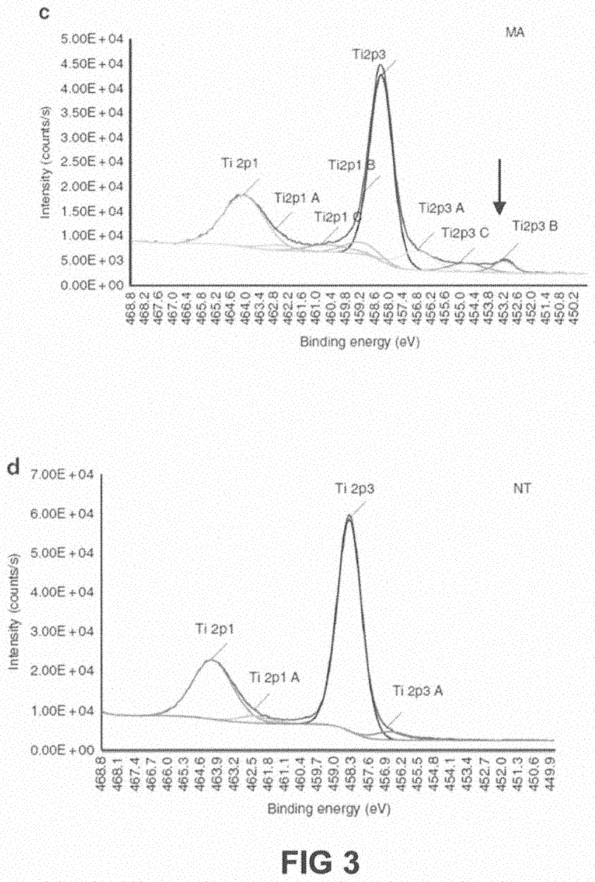

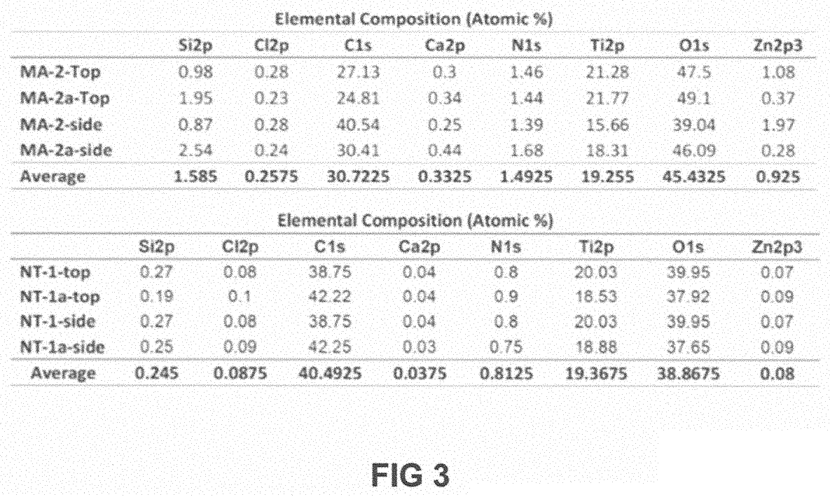

To test whether the complex grit blasting, acid etching and nano-tube creation on the NT surfaces induced chemical differences between MA and NT surfaces, we analyzed the elemental composition of the lateral and top surfaces of each type of Ti implant by X-ray photoelectron spectroscopy (XPS). Survey spectra of MA and NT samples are shown in (FIGS. 3a and b) and Table 1 lists the relative atomic percentage for each of the elements labeled in FIGS. 3a and b. The same 3 predominant peaks, O1s, Ti2p and C1s are visible in the survey spectra for MA and NT surfaces with no discernable distinctions in the minor peaks—N 1s, Ca 2p, and Si 2p. Since C1s is from adventitious carbon the only two relevant elements are Ti and O. Since the O1s envelope will have contributions from C—O groups (note the increase in O1s in the MA group is inversely proportional to the decrease in C1s, with respect to the NT group), we have only focused on the Ti envelop in our deconvolution.

Thus, high-resolution Ti 2p spectra were obtained to compare the nature of the TiO₂ oxide layer (FIGS. 3c and d). The 2 dominant peaks in the spectra of the both implant surface types are due to Ti2p1 and Ti2p3 which can be assigned to TiO₂²⁶. The only additional peak visible in the MA sample is that for Ti2p3B—the emission due to the underlying titanium metal. The absence of this peak in the NT sample shows that the oxide layer is sufficiently thick to prevent electron emission from the underlying metal. Its presence in the MA sample indicates that the oxide layer is sufficiently thin to allow electron emission from the underlying metal. Since, in XPS, the penetration depth of electrons is a maximum of 10 nm, it means the oxide layer on the MA surface is less than 10 nm but greater than 10 nm on the NT surface.

Importantly, while we did not detect significant chemical differences between the MA and NT surfaces, they do exhibit differences in their TiO2 surface oxide layer thicknesses and the topographical differences were obvious as observed by FE-SEM.

### μCT Imaging of Peri-Implant Bone Formation

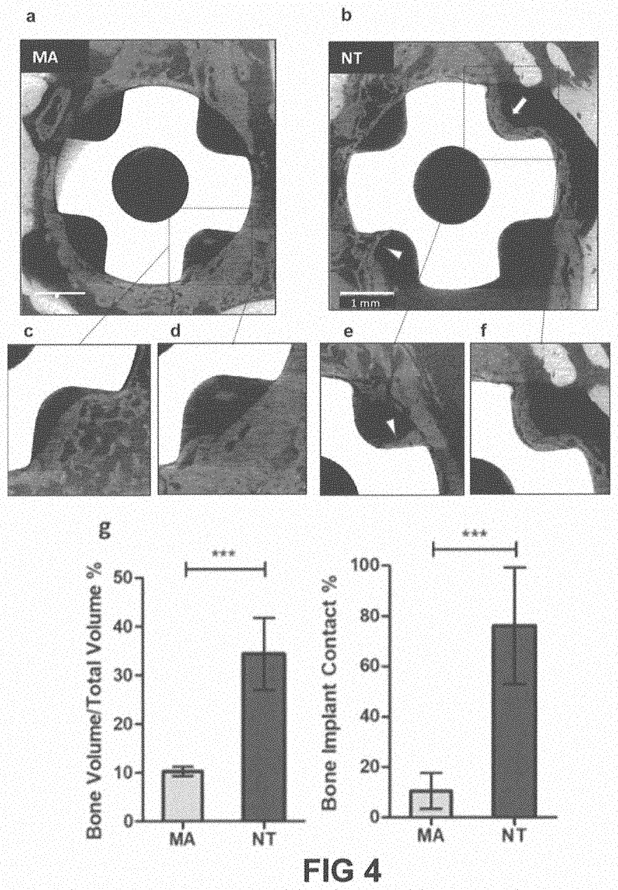

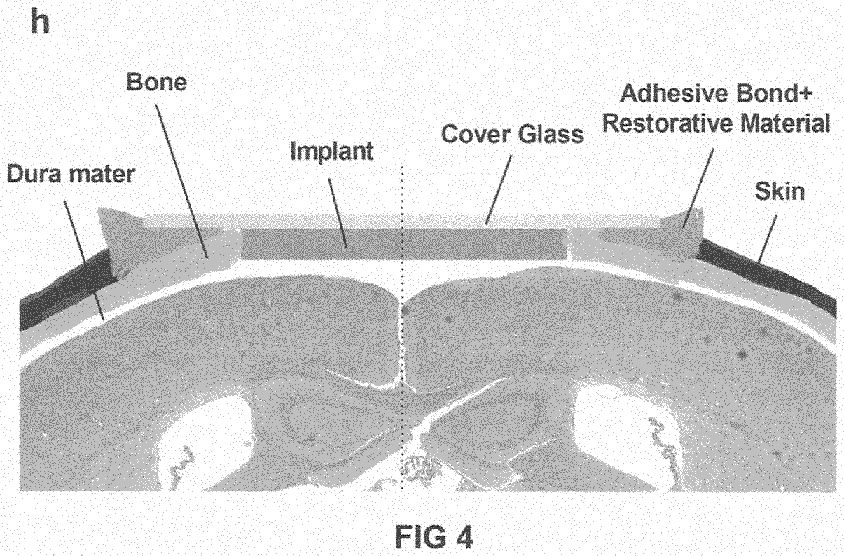

Samples of the entire skull from both implant groups were scanned at 2, 4 and 6 weeks after implantation using microcomputed tomography (μCT). No bone was detected in the healing volumes at week 2 in the NT group (Supplementary FIG. 1A). At the end of week 4, contact osteogenesis was observed on the NT implant (Supplementary FIG. 1B), but not the MA group. The μCT scanning of the entire defect area in both groups at week 6 showed that the new bone had been formed in different locations depending on the implant surface topography: on the edge of the craniotomy defect (distance osteogenesis) in MA group (FIG. 4a), but directly on the surface of the NT implants (FIG. 4b). Individual μCT scans, at 2 different depths, clearly showed new bone growth into the healing volumes of MA was initiated at the craniotomy margin (FIGS. 4c and d), while the NT samples exhibited osteoconductive bone formation either as a seam of bone on the implant surface or the ingress of bone along the implant surface as characterized by a Baud curve (FIG. 4e,f and Supplementary Movie 2). Quantitative comparison of the bone volume over total volume (BV/TV %) and bone implant contact (BIC %) showed a significant increase in the amount of bone formed in the healing volume and on the surface of the implant in NT samples FIG. 4g. Schematic in FIG. 4h shows the coronal view of the CIWC in the craniotomy.

### In Vivo Imaging of Neovascularization in the Peri-Implant Wound Site

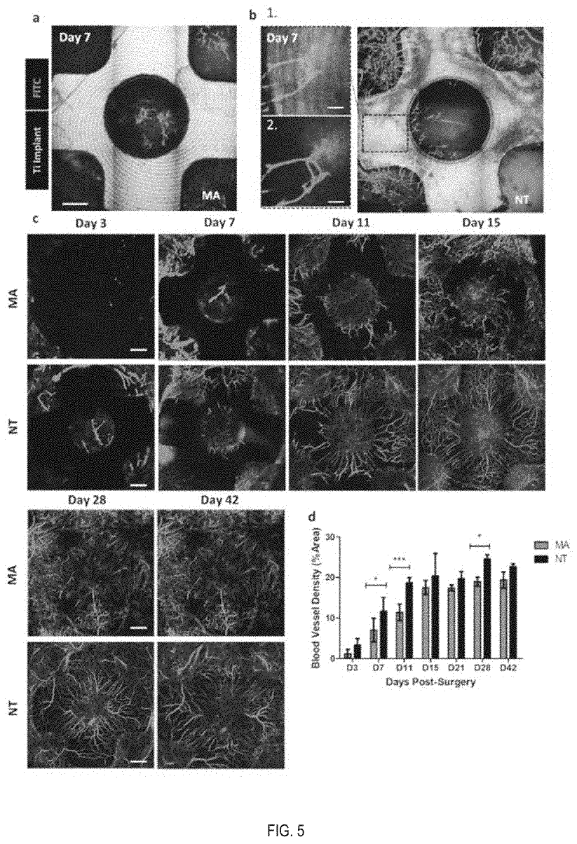

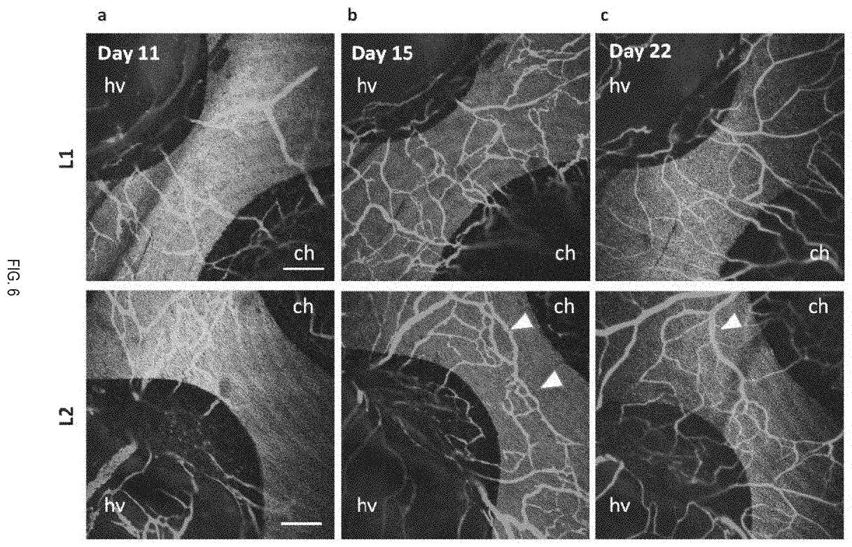

The spatio-temporal dynamics of peri-implant wound healing were examined in vivo in C57BL6 mice using our experimental CIWC model. The CIWC remained durable, and infection-free, for up to at least 6 weeks. The CIWC permitted intravital longitudinal tracking of neovascularization at the peri-implant wound site by confocal fluorescence microscopy. Vessels were visualized by tail vein injection of a high molecular weight (2 MDa) fluorescein isothiocyanate-dextran (FITC-DEX) that had a low extravasation rate in intact vessels. Development of the vasculature in the peri-implant healing site was tracked from day 3 to 42 post-implantation. Neovascularization occurred earlier around the NT surface than the MA surface, and extravasated FITC-DEX was mostly visible from the vessel tips at earlier time points (FIGS. 5b.1 and 5b.2). FIG. 5c shows representative images of vascular development over a period of 42 days around both MA and NT implants. In the MA group, negligible fluorescence signal was detected within the craniotomy defect at day 3. By day 7, some vessels were observed at the periphery of the defect, and within the central implant hole, with evidence of extravasated FITC-DEX. Between days 7 and 11, vessels grew over the top of the MA implant surface. Between days 11 and 42, vessels grew in length and while some vessels partially anastomosed, the majority remained fragmented with a disorganized pattern. On the contrary, both the rate and pattern of vascular development around the NT implants were different. More vessels had been formed at day 3, by day 11 the vessels had grown over the top surface of the NT implant, anastomosed and formed a dense network. This network was more organized compared to the MA group by day 15, exhibiting a less tortuous, predominantly radial and more evenly distributed spatial pattern. Larger vessels were apparent by day 28 and at day 42.

Comparative analysis of the functional vessel density^{28,29}, from weeks 1 to 6, was quantified (as % fluorescent area of each defect) by keeping the concentration and administration dose of FITC-DEX the same in both implant groups, and across all imaging time points (FIG. 5d). Longitudinal fluorescence imaging data showed that the functional vessel density in the NT group was higher than the MA group at all time points. This difference was significant at the earlier time-points, days 7 and 11, and also at day 28—increases of 66.78%, 64.5% and 30.1% respectively. This quantification of the blood vessel density was consistent with the known phases of vascularization—progression, regression, and remodeling—visualized in FIG. 6a-c for two distinct fields-of-view, from days 11, 15 and 22, in NT implants.

### The Assessment of Neovascular Morphogenesis

To characterize the morphology of the vasculature developed proximal to the implants at early time points after implantation, morphometric analysis was performed on the confocal intravital images of the FITC-DEX taken at days 7 and 15 post implant surgery. An example of an healing volume from each of the MA and NT groups, respectively, is shown in FIGS. 7a and b. The greater degree of neovascularization in the NT group is evident, while the less well-developed vessels around the MA group show a more leaky appearance. FIGS. 7c and d show the vascular skeletons corresponding to FIGS. 7a and b, which were used to identify the vessel segment coordinates and measure the following vascular morphometric parameters: vessel branching number, vessel volume, and vessel length. The vessel density and vessel length are a measure of the quantity and the continuity of the vessels, respectively. The vessel volume is a 3D fluorescence-based measurement of the entire vascular volume in the pen-implant wound site occupied by intravascular FITC-DEX, which provides an estimate of the total blood volume in the wound site. According to the box plots in FIGS. 7e and f, the NT vessel segment coordinates were significantly higher than those of the MA implants in both axes (25% and 30% increase in the median values for X and Y coordinates respectively). The number of vessels within the top quarter percentile is higher in NT compared to MA implants as can be seen from both the range, and number, of data points within the top quarter percentile. As the top quarter percentile is the closest spatial region to the implant lateral surface, these data show that the number of vessels in proximity to the NT surface was significantly higher than seen with the MA surface (p<0.0001 for both X and Y coordinates).

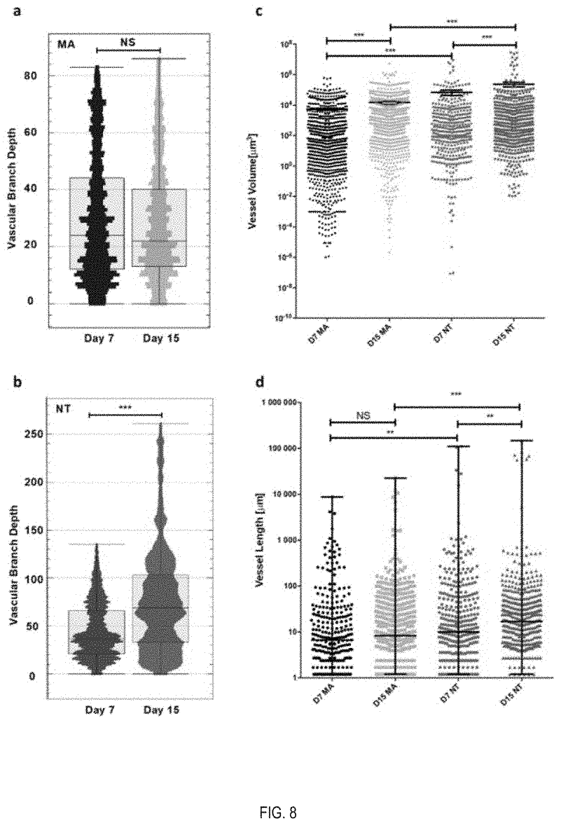

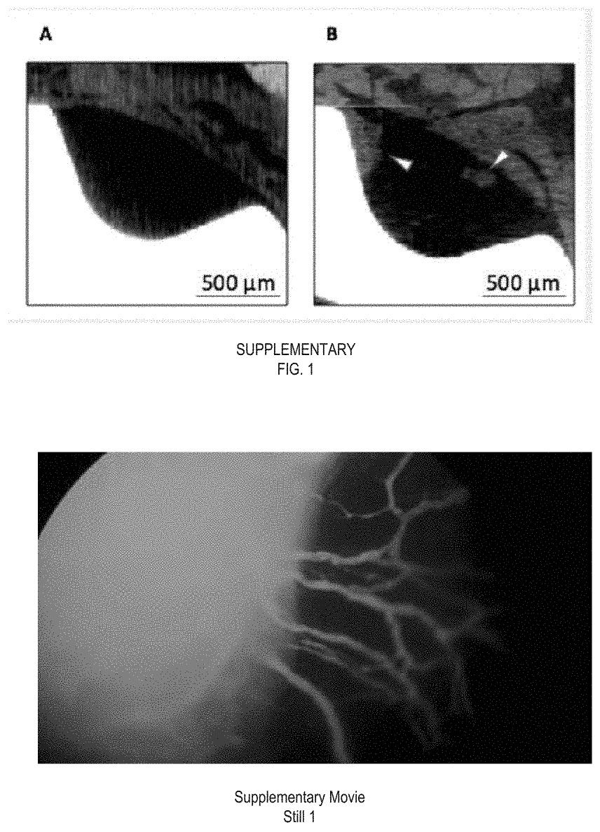

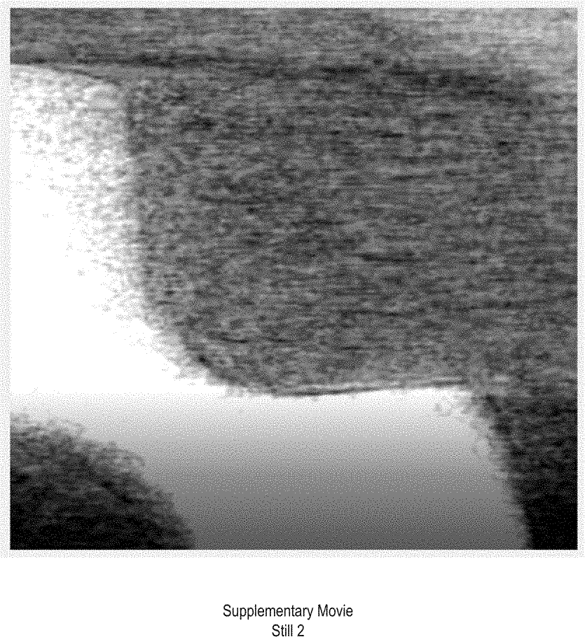

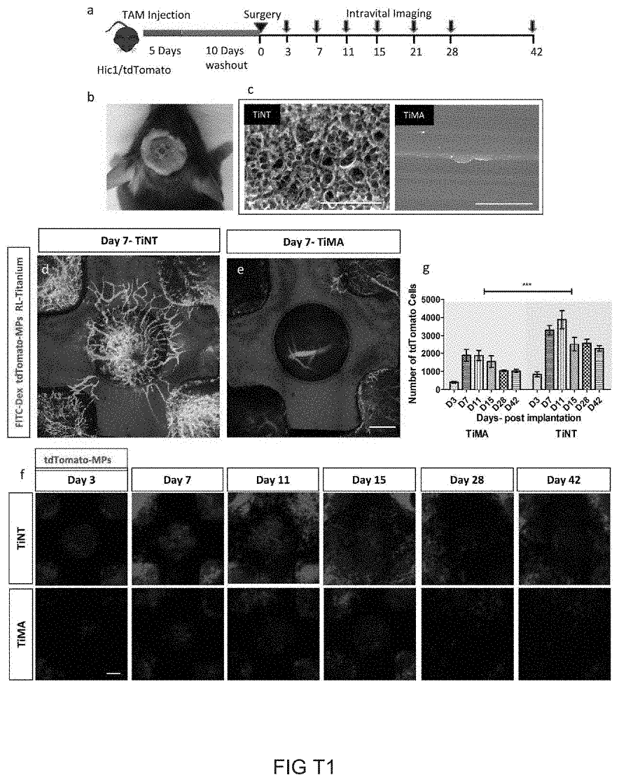

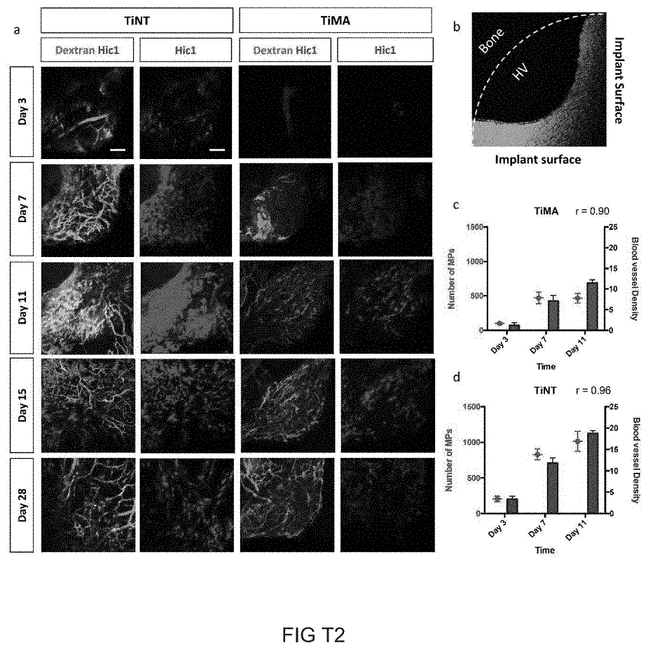

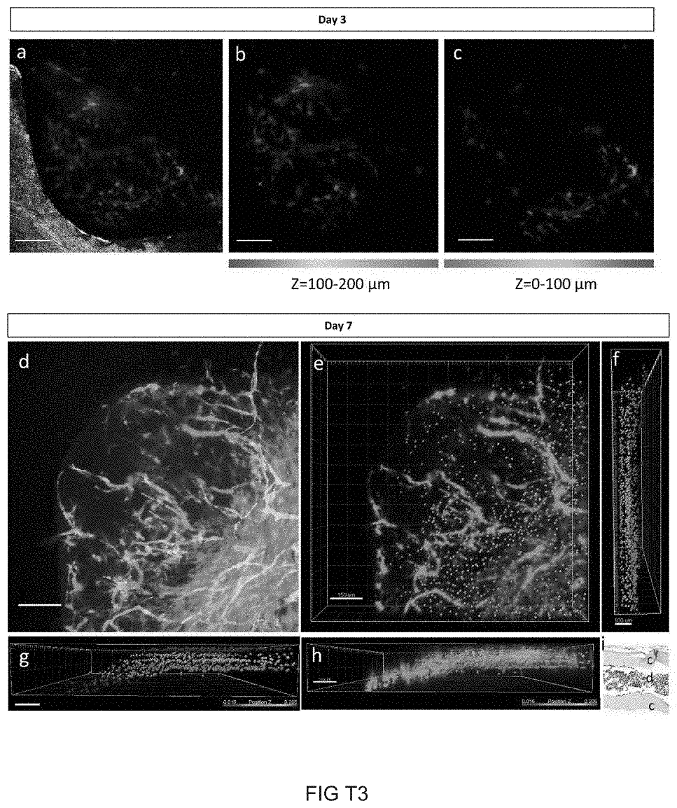

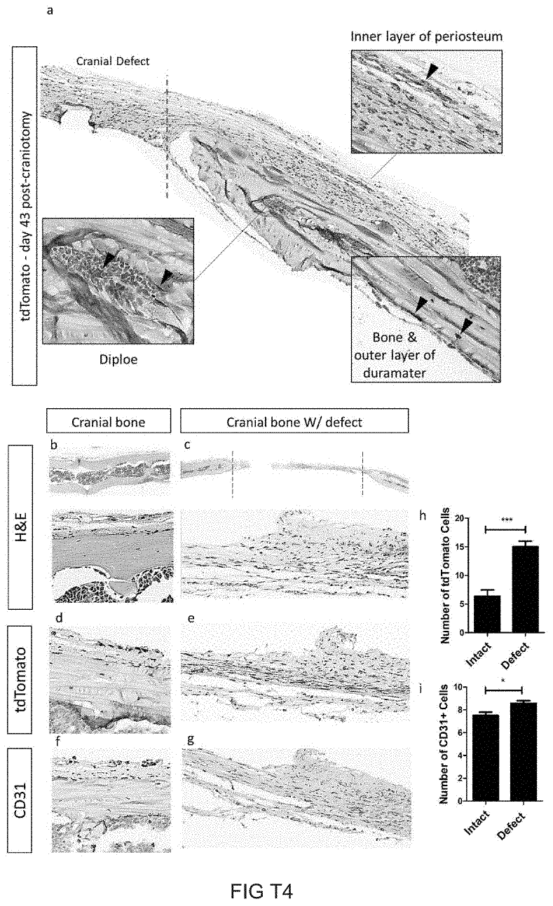

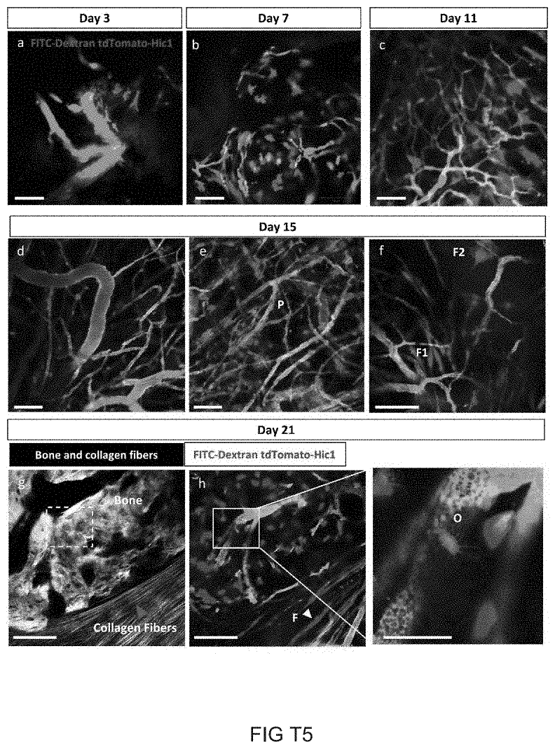

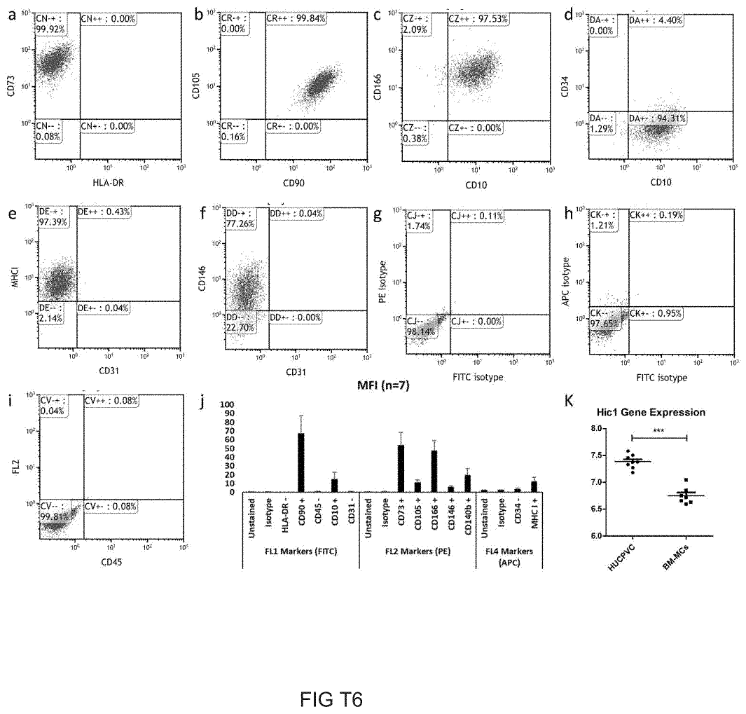

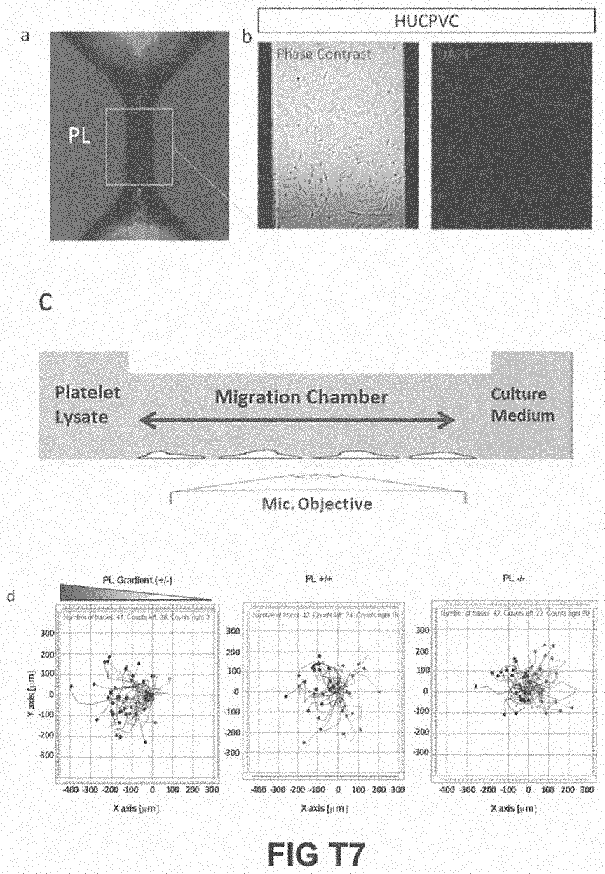

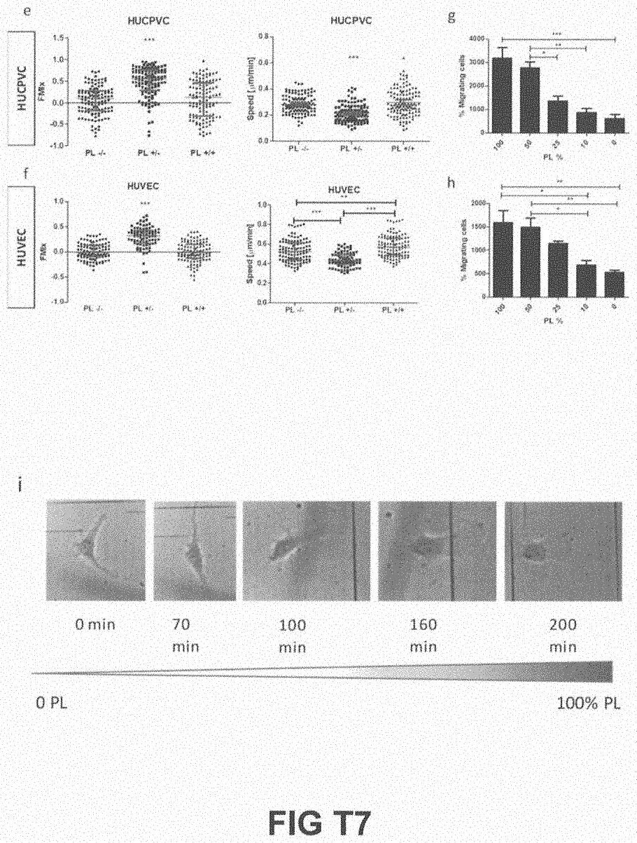

At day 7, the NT group exhibited a hierarchically branched network of the vessels with small branches that grew over the surface of the implant and distributed along the lateral surface (FIG. 7d). The vessel branching number which is a measure of vessel sprouting in a developing microvascular network, was significantly higher in the NT (136) than the MA group (83), at day 7 (FIG. 7g). The vessel branching number did not change from day 7 to day 15 in the MA group (FIG. 8a), while there was a 92.5% increase of the maximum in the NT group, with a median increase of 89% (FIG. 8b). Assessment of the vascular network volume, which represents the volume of the blood flow within the peri-implant wound site, showed a significant increase in both implant groups between week 1 and 2 (FIG. 8c). However, at both days 7 and 15, the mean vascular volume was significantly higher around the NT surface (69,858 and 240,440 μm³ respectively) compared with the MA surface (5,422 and 14,575 μm³, respectively). A similar trend was observed in vessel length data (FIG. 8d), with no significant difference between weeks 1 and 2 in the MA group. The distribution of the vessel length around the NT implant at day 7 was similar to day 15, ranging from very short branches to long branches. However, the fold increase (106%) in median length sum by day 15 in the NT group is suggestive that the shorter branches have been remodeled to form longer branches.

### Discussion

While neovascularization is an essential prerequisite to osteogenesis, no previous published reports have examined the effect of implant surface topography on the spatiotemporal pattern of neovascularization during endosseous peri-implant healing in vivo. Our results clearly show that the surface design of the implant has a profound effect on the pattern of neovascularization with new vessels being developed at, or near, the implant surface and the vascular network maturing through remodeling sooner in the presence of a topographically complex surface. The rapid development of a functional vascular supply is of key importance to pen-implant wound healing, both as a source of scavenging and immune-modulating leukocytes, and a nutrient supply to support tissue regeneration. Indeed, the rate of osseointegration is critically dependent upon osteoconduction—the key determinant of contact osteogenesis³⁰—and we have shown, quantitatively, that this can be accelerated by increasing the topographic complexity of the implant surface³¹. Thus, our findings provide a new perspective on the importance of implant surface design that is relevant to many therapeutic areas including orthopedics, dentistry, otorhinolaryngology and plastic surgery. Previous studies have established the window chamber model as a tool to longitudinally image the spatia-temporal development of both neovascularization and osteogenesis in craniotomies^{32,33}. An observation common to these, and microCT, calvarial studies is that new bone grows centripetally within the bony defect both in the un-modified state^{34,35} or when the defect is modified by the addition of growth factors^{34,35}, cells³⁶ or cells and scaffolds³⁶⁻³⁸. This is important because we show, on the contrary, that when a metallic implant is introduced into such a model, the pattern of bone growth is modulated as a function of implant surface topography: the MA (smoother) and NT (rougher) surfaces exhibited distance and contact osteogenesis respectively¹. This observation provides an essential validation of our CIWC model as it has been generally accepted, since the work of Buser et al (1991)¹², that implant surface topography has a profound effect on contact osteogenesis. Indeed, we have established the functional significance of three distinct scale ranges of implant topography on both bone bonding and bone anchorage, two distinct mechanisms within the phenomenon of osseointegration³⁹. The current study investigated the effect of implant surface topography on peri-implant neovascularization using two surfaces, a relatively smooth machined (MA) surface and a complex microtopographic surface with superimposed nanotubes (NT). However, our platform would be suitable for studying spatia-temporal vascular morphogenesis around other surfaces beyond those discussed in the present paper.

Our model has enabled direct visualization of three distinct phases of vascularization during the first 42 days of healing: capillaries sprouted and grew longer, anastomosed to form loops and, finally, remodeled into a more functional vasculature that facilitated blood flow throughout the peri-implant site. High-resolution images showed that the vasculature grew predominantly from the periphery of the bony defect towards the lateral surface of the implant, but vessels also grew from the dural surface into the central implant hole. With time this peripheral and central vasculature anastomosed on the top flat surface of the implant, with blood flow in each direction (Supplementary Movie 1). Although such anastomoses occurred on both the machined and topographically complex surfaced implants, only the latter displayed an ordered, radial, arrangement of vessels, a pattern completely absent on the machined surface, during the time course of our experiments. Indeed, we demonstrated that the NT surface not only increased the rate of neovascularization following endosseous implantation, but also changed the morphological characteristics, spatial pattern, and functionality of the re-established microvasculature. Interestingly, while the machining marks were obvious on the machined implant, they were less evident on the complex surface. There have been numerous reports of cell migration along the long axes of surfaces with linear features^{40,41} but we saw no evidence that these topographic features influenced the directional growth of vessels.

At the earliest days of healing, in both implant groups, the neovessels were highly permeable as FITC-DEX extravasated from both the lumen and ends of the nascent vessels, appearing as a bloom of fluorescence. With time, and increasing function, extravasation of the FITC-DEX through the vessel walls was reduced and only leaked out from the vessel tips. We believe that such extravasation is due to the immaturity of the distal blood vessels, since it was absent at later time points.

Morphological properties of the microvascular system affect the blood flow and its distribution within the wound area⁴². The morphometric parameters used in this study which were measures of vascular density, volume, length and branching number are indicators of the ability of the vasculature to distribute flow throughout the tissue. These are standard parameters used by several studies assessing physiological^{32,43} or pathological angiogenesis^{44,45}, although representation of the data on combined box/whisker and scatter plots provides additional graphic information concerning the frequency distribution of the individual vessels in the complex 3D network.

From the physiological standpoint, distribution and collection of blood-borne substances within tissues and organs requires a branching system. Hierarchical branching of a vascular network—starting from a relatively large stem vessel to smaller and smaller branches—is essential for conducting flow further into the wounded area. However, a non-optimal vascular density reduces vascular functionality⁴⁶. Therefore, the pruning of excessive vessels is essential for maturation of a vascular network. The branching number shows increased branching around NT implants compared to MA implants. The early dense network of small vessels matures, through remodeling, to larger functional vessels that conduct a higher volume of the blood. During the maturation of the vascular network some of the morphological features such as vessel length, volume and branching change concomitantly as there are scaling relations between these parameters⁴⁷. The choice of one vessel over another in the pruning process, is known to be based on blood flow⁴⁸. Vessels with higher blood flow increase in girth while those with lesser blood flow regress. Our results show a higher mean vessel volume in the NT group both at week 1 and 2 compared to the MA group. However, the number of vessels is higher in the MA group. This indicates that large vessels have an essential role in increasing the bulk flow compared to numerous small vessels. By week 4, the vascular network was remodeled to form larger vessels that improved functional blood flow for both implant types. This measure of blood flow is important since it has been reported that the progenitor cells position themselves relative to the volume of the blood⁴⁹, and vessels were consistently larger around the NT implants.

Since we would not expect to image vessels that may have formed independent of the pre-existing vascular network, as they would not be labeled with FITC-DEX unless they had anastomosed with those that had developed from the functional vasculature of the circulation, we cannot exclude the possibility of vasculogenesis as distinct from angiogenesis⁵⁰. However, our results do show that the changing characteristics, structural organization, and spatial location of the re-established vascular network around the two implant surfaces was reflected in a corresponding change in the spatial pattern of bone healing. Previous cranial defect healing models have suggested that the osteogenic precursor cells can originate from the periosteum, bone marrow (BM)^{51,52} and dura matter⁵³, and we would expect these tissue-resident mesenchymal cells, to be of perivascular origin⁵⁴ although not pericytes⁵⁵. In fact, Hung et al. showed that there is a correlation between the morphometric characteristics of the vascular network, particularly the diameter and the length of the blood vessels and the volume of the differentiated osteoblasts in their vicinity⁵⁶. Thus, by altering the surface characteristics of the implant, which we have shown to have profound effects on neo-vascularization, the ingress of osteogenic precursors and their location with respect to the implant surface is also being affected, resulting in either contact or distance osteogenesis.

In contradistinction to previous reports, our model provides a unique and reproducible preclinical platform to study implant healing biology over clinically relevant time scales. The window is durable for more than 6 weeks, sufficient to monitor early critical stages of both peri-implant neovascularization and osteogenesis. Using intravital imaging, we obtained both qualitative and quantitative information on the complex 3D structure of the neovascularization with respect to the two different implant surfaces over a large region of interest (4 mm). Tracking active vascularization from initiation to remodeling in a single animal, over multiple time points, reduces animal-to-animal variation and increases the reliability of the quantification. Interestingly, the presence of the implant blocked much tissue auto-fluorescence and resulted in an increased signal-to-noise ratio. Together with longer pixel dwell, these details may account for the higher resolution images we obtained compared to previous intravital studies^{32,33}. Titanium-based implant materials are commonly employed in orthopedic, craniofacial and dental surgery due to their combination of mechanical properties, corrosion resistance and biocompatibility⁵⁷⁻⁵⁹. Our results show that a topographically complex surface contributes to the development of a radially arranged vascular structure with hierarchical branches spatially closer to the surface of the Ti-implant. These findings emphasize the translational importance of a rationale for implant surface design, which could help improve the clinical effectiveness of endosseous implants compared to traditional implant surfaces. As neovascularization is the route for the ingress of both immune and progenitor cells, alterations in the surface topography would enable healing through regulation of neovascularization. A comprehensive understanding of the healing and regeneration mechanisms of endosseous integration in the pen-implant niche has a considerable impact in implant medicine. The knowledge transferred from the current study provides one step forward towards designing endosseous implants capable of controlling endogenous peri-implant vascularization.

### EXAMPLE 2

We have recently developed a cranial implant window model⁴ with which we have longitudinally tracked the spatia-temporal development of peri-implant neo-vasculature using intra-vital microscopy⁵. Using this model, we have shown that the pattern of angiogenesis in the wound site can be profoundly, and reproducibly, influenced by the surface topography of a metallic implant. Since angiogenesis precedes osteogenesis in bone wound healing, the model enabled us to demonstrate that the pattern of peri-implant angiogenesis determined that bone formed in contact with a topographically complex (TiNT), but distant from a smoother machined (TiMA), titanium implant surface. However, the means by which the mesenchymal osteoprogenitors populated the wound site were not examined.

Now, using the same implant surfaces, applying our imaging model to a Hic1 (Hypermethylated in Cancer-1) mouse model, in which perivascular mesenchymal progenitor (MPs) cells are labeled with a fluorescent protein (tdtomato), has allowed us to longitudinally track MP migration, the differentiation of their progeny, and their spatiotemporal relationships to neo-vascularization of the wound site.

Hic1 is a gene involved in craniofacial development^{6,7} in both human and mouse and marks a broad population of perivascular mesenchymal progenitor cells⁸. The Hic1 marker extensively overlaps with PDGFRα and Sca1⁸, which are common markers of mesenchymal precursors in various tissues⁹⁻¹¹. PDGFRα is a mesenchymal marker in both human and mouse; and it was recently found that PDGFRα⁺ cells that reside in injured peripheral nerves are mesenchymal precursors that can directly contribute to digit tip regeneration and skin repair in mouse¹². On the contrary, Sca1 is a marker of hematopoietic and mesenchymal cells unique to mouse. Thus, we assessed the impact of implant surface topography on MP ingress into the pen-implant healing compartment.

Increasing implant surface topographic complexity results in enhanced platelet activation¹³ and consequent signaling. We hypothesized that the migration of both perivascular and endothelial cells could be driven by the differential activation of platelets on the implant surfaces employed. To test this hypothesis, we undertook in vitro modeling to interrogate the migratory behavior of both Hic1⁺ and endothelial cell populations in the presence of a linear density gradients of human platelet lysate (PL).

### Results

Intravital Imaging Reveals that Complex Implant Topography Enhances Recruitment of Mesenchymal Progenitor Cells to the Implant Surface. (FIG. T1)

We assessed the dynamics of peri-implant MP ingress by repeated intravital imaging. We studied the behavior of MPs intravitally in mice containing tdTomato reporter knocked into the Hypermethylated in cancer (Hic1) gene. We crossed a (Hic1-CreERT2) knock-in line with RosaLSL-tdTomato mice. Tamoxifen injection for five consecutive days gave rise to strong Hic1-specific expression of tdTomato. After a 10-day wash-out period, live imaging was performed according to the timeline illustrated in FIG. T1a. Implantation of the cranial implant window chamber (CIWC) was performed according to the previously established method¹⁴ (FIG. T1b). Machined (TiMA) and topographically complex nanosurfaced (TiNT) implants (FIG. T1c) were used to investigate the influence of implant surface on MP dynamics and behavior.

FIGS. T1d and e are representative images taken through the window chamber implanted within the calvaria of Hic1/tdTomato mouse. Three fluorophore channels were imaged by confocal microcopy; endogenously fluorescent MPs (tdTomato) in red, new functional blood vessels visualized by tail vein injection of high molecular weight (2 MDa) fluorescein isothiocyanate-dextran (FITC-Dex) in green, and the titanium implant (silver gray) within the bone. Images were collected in tiled z-stacks of the entire implant wound area, which contains a titanium implant placed in a 4 mm in diameter craniotomy. FIG. T1d shows the infiltrating MPs, and the developing vasculature around a nano surface (TiNT) implant. Dramatic differences in the regeneration activities are visible compared to the TiMA surface (FIG. T1.e). The spatiotemporal dynamics of MPs around both TiMA and TiNT implants was tracked from day 3 to 42 post-implantation (FIG. T1f). At 3 days post-implantation, tdTomato cells were observed in each of the healing volumes around the TiNT implant. At day 7, a substantial increase in the number of the cells (2.9 fold) was observed which continued until day 11 but started to diminish from day 11 to day 43 post-implantation. In the TiMA group, a few tdTomato cells were observed at the wound area on day 3, this number gradually increased from day 3 and day 15 and declined from day 15 to day 42. 3D quantification of the number of MPs in the fluorescence images from weeks 1 to 6 following implant placement surgery validates the trend observed in the longitudinal images (FIG. T1g). Two-way ANOVA confirmed that time, and implant surface topography, significantly affect the number of tdTomato cells recruited to the wound site (P-value<0.0001).

Mesenchymal Progenitor Cells are Perivascular, but not Pericytes, and Their Entry in the Wound Site is Correlated with Angiogenesis (FIG. T2, T3a)

To elucidate the events occurring within the bone-implant wound site, we closely looked at one of the healing volumes in the cruciate shaped implant. We performed intravital longitudinal imaging on Hic1/tdTomato mice from day 3 to 42 post-implantation and tracked the healing events in the proximity of the implants with nano (TiNT) and smooth (TiMA) surfaces (FIG. T2a). Red and green channels show tdTomato MPs and FITC-Dex neovasculature respectively. FIG. T2b is the corresponding reflected light channel visualizing the Ti implant, showing the region of interest that has been imaged over time in both implant groups.

Longitudinal observation of tdTomato expression demonstrates dynamic changes in the population of MPs over time. What was remarkable here was the emergence of a proliferative bloom of tdTomato⁺ cells in the TiNT group at day 7 post-implantation which peaked at day 11, and diminished by day 28. Simultaneous imaging of the MP cells and the blood vessels suggests an association between the population of the MPs in the wound site and the growth of the new vessels. Interestingly, the population of tdTomato progenitor cells and blood vessels was remarkably lower at all early timepoints in the TiMA group compared to the TiNT group. The appearance of leaky vessels indicates slower development and maturity of blood vessels at day 7 post-implantation in the TiMA group. The absence of the bloom of tdTomato cells in the periphery of the TiMA implant is an indication of lower regenerative activity. The early progression of neo-vascularization clearly visualized at Day 7 around the TiNT implant was only seen at Day 28 in the TiMA samples, at which time vascular remodeling around the TiNT samples was evident.

The quantification of functional vessel density and the number of the MPs present in a healing volume at early timepoints (day 3 to day 11) for both implant groups (FIGS. T2c and d) is consistent with microscopic observations. The Pearson correlation coefficient (r=0.9 for TiMA and 0.96 for TiNT) indicates a positive correlation between the growth of the new vessels and the abundance of MPs in the wound site. Moreover, the number of tdTomato cells and functional blood vessels approximately doubled in the TiNT implant wound healing site compared to the smooth TiMA implant.

Despite extensive evidence of an association of mesenchymal progenitor cell recruitment with wound site vascularization, it appears that these perivascular cells are not bound to the newly growing vessels in the first few days post-implantation. In FIG. T3a we split the Z stack images of a 3 day sample into two compartments: the body of the wound and thus closer to the glass cover-slip (Z=100-200 μm) (FIG. T3b) and the deeper compartment close to the dura mater (Z=0-100 μm) (FIG. T3c). Since the dura mater was not subject to injury, the blood vessels in the dural tissue remain intact and stable, with pericytic coverage. However, in the body of the wound the abundant perivascular cells are not bound to the new vessels as pericytes.¹⁵

### Hic1 Expressing MPs are Located in Vascularized Areas of the Cranium and Show Multipotency During Defect Healing (FIGS. T3b and T4 and T5)

3D spatial analysis of red (Hic1⁺ MPs) and green (blood vessels) fluorescence channels was performed to identify the microanatomical location of activated MPs in the peri-implant wound site. 3D reconstructions of each implant healing volume were created as demonstrated in FIG. T3d-h. tdTomato MPs were detected using the spot detection algorithm in Imaris. Spots were overlaid with blood vessels to show the relative location of MPs. FIG. T3f-h shows the sagittal and coronal views of the representative healing volume. The flat bone which surrounds the implant is composed of two cortical bone plates between which is the diploë (FIG. T3i). The outer surface of the cranium is covered by the ecto-cranial periosteum, while the dura mater serves as the endocranial periosteum. Taking this into consideration, FIG. T3g shows that the majority of MPs were localized in the inner layer of the ecto-cranial periosteum. Although the outer layer of the periosteum was removed from the bone during the placement of the CIWC, it is likely that cells from the inner layer remained on the bone surface and migrated towards the implant surface in response to injury. 3D spatial visualization of MPs overlaid with blood vessels in the coronal optical section provides clear evidence that MPs were localized within the perivascular niche and follow the exact trajectory of blood vessels although not directly bound to them.

The above discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

We identified the original location of the tdTomato/Hic1 positive cells within the cranium and compared the quantity of these cells and the vasculature in injured versus intact cranium. For both conditions, histology confirmed the presence of the tdTomato cells in the periosteum, diplöe, and dura mater, but the majority of MPs were visible in the inner layer of periosteum (FIG. T4a) (marked by arrow heads). Representative images of the consecutive sections stained for haematoxylin and eosin (H&E), red fluorescent protein (RFP), and cluster of differentiation 31 (CD31) in both intact and injured crania are shown in FIG. T4b-g. Intense positive staining for RFP in the injured model indicates enhance contribution of MPs in response to injury (FIG. T4e). Quantification of RFP positive cells in the defect model shows a 3-fold increase in the number of MPs compared to the non-osteotomized controls (FIG. T4h). However, there is no significant difference in vascularization between the defect and control at day 42 post-implantation, as demonstrated by CD31⁺ cell quantification (FIG. T4i). This would indicate that vascularization reaches homeostasis at this timepoint and the vessel density reached that of a normal tissue. However, the higher number of tdTomato MPs in the defect model suggests that these cells might be off the vessels and actively differentiating into other lineages.

Intravital Microscopy (lVM) on single cells in the cranial defect at various timepoints during healing identified phenotypically distinct cells labeled with tdTomato (FIG. T5.a-h). At day 3 and 7 post-implantation tdTomato cells seem mesenchymal/fibroblastic in morphology, as fibroblasts normally reside in the interstitium, unbound to blood vessels (FIGS. T5a and b). At day 11, some of the tdTomato cells appeared expanded and intensely fluorescent, while others stabilized on blood vessels in a pericytic manner. At day 15, various morphologically distinct cells were visible in the wound niche expressing tdTomato (FIG. T5d-f). Among these are an abundance of pericytes visible in different regions of interest (ROIs) circumferentially wrapping around the capillaries. A typical pericyte, marked “P” in FIG. T5e is closely juxtaposed to the vessel wall with cell processes enveloping the vessel. Fibroblast-like cells were also visible on day 15, showing a migratory morphology (FIG. T5f). These cells appear as either ‘F1’, long and spindle-shaped and migrating within the 3D matrix, or “F2”, flattened on the matrix with a leading edge and trailing tail (as it can be observed in cell culture). The addition of the SHG channel allowed visualization of bone and collagenous matrices at later timepoints, (e.g. day 21 post-implantation: FIG. T5g). The white areas are bone and blue arrow heads indicate the fibrous tissue. Overlaying the SHG image with red (tdTomato) and green (FITC-Dex) fluorescence channels showed osteocytes buried with the bone. In the magnified region of FIG. T5h, “O” is a tdTomato osteocyte with long cell processes buried within the bone.

### Human Umbilical Cord Perivascular Cells are a Characterized Human Source of Hic1⁺ Mesenchymal Progenitor Cells

Human umbilical cord perivascular cells (HUCPVCs) were used for all in vitro experiments as a characterized population of perivascular cells. These cells are a non-hematopoietic population of cells isolated from the perivascular tissue of the cord that are capable of differentiating into myogenic, adipogenic, chondrogenic, and osteogenic lineages in vitro¹⁶. These cells have a fibroblast-like morphology with a stellate shape and long cytoplasmic processes. Flow cytometry showed the expression of MSC surface markers CD105, CD90, CD73 CD166, CD146, CD 140b/PDGFRb, CD10 and MHC1. Also, these cells lacked the expression of hematopoietic lineage markers CD34, CD45 and HLA-DR and endothelial cell markers CD31 (FIG. T6a-i). Mean Fluoresce Intensity (MFI) of each panel showed the level of expression of each of the surface markers mentioned above (FIG. T6j). To validate that HUCPVCs are a rich source of Hic1, gene expression analysis was carried out on passage 3 HUCPVCs. The gene expression analysis was performed in comparison with human bone marrow mesenchymal cells (BMCs) (FIG. T6-K).

### A Gradient of Platelet Lysate Controls Motility and Coordinates Migration of Perivascular Mesenchymal Progenitor, and Vascular Endothelial, Cells

Our previous in vivo assays showed that the nanosurface changes the pattern of neovascularization in, and the current findings the recruitment of MPs to, the peri-implant compartment. The cause of this differential pattern is currently unknown. However, platelets activated on the implant surface release multiple growth factors and cytokines (i.e. PDGFα, PDGFβ, VEGF-A and TGFβ) in high concentrations and provide ligands for the cells that reside in the wound niche. Therefore, we conducted in vitro tests for the real-time chemotaxis and migration of endothelial and Hic1⁺ MPs in response to a linear gradient, at various concentrations, of human platelet lysate (PL). Both cell types adhered to the bottom of the slide (FIGS. T7.a and b) and were exposed to either a diffusible linear gradient of PL (+/−) (FIG. T7c), a gradient-free concentration of PL (+/+), or serum-free culture medium (−/−). Live-cell-imaging of the observation area sequentially at 10-minute intervals allowed trajectories of single cells to be obtained in every condition for 24 hrs. As shown in representative trajectories of the perivascular cells (FIG. T7d), when introduced to a gradient of PL, cells migrate towards the highest concentration of PL, although negligible indirect movements always existed due to random walk. In control experiments, with or without PL, no preferential pattern was observed in the migration of the cells. Measuring forward migration index (FMI) and motility speed for perivascular cells showed a significant increase in the forward migration index in the PL gradient condition compared to both positive and negative controls (FIG. T7e). Likewise, the endothelial cells showed a similar migratory pattern (FIG. T7f). Since endothelial and perivascular cells are dispersed in the wound site, and they make random movements in all directions, the measured directionality means the average movement of the cell population is directed toward the implant surface. Transwell migration assays also showed PL stimulated migration of perivascular and endothelial cells in a dose-dependent manner, reaching an approximate plateau by a concentration of PL higher than 50% in serum-free medium (FIGS. T7g and h).

### Discussion

In this study we showed that the microvascular bed contains resident mesenchymal progenitor cells that directly contribute to post-surgical tissue repair and regeneration. We used IVM to anatomically and functionally map blood vessels and associated mesenchymal cells. We first showed that the Hic1⁺ MP cells populate the wound site rapidly to form a dense “bloom” of proliferating cells which appear in areas demarcated by the neo-vasculature but show no preferential juxta-positioning to the vessels themselves. Interestingly within the time frame of 11 to 28 days, the number of the tdTomato labeled Hic1⁺ cells diminishes but an increasing number of these cells are found in intimate contact with the vasculature displaying a pericytic morphology with cell processes wrapping around individual vessels or vessel junctions. Similarly, other Hic1⁺ cells exhibit either a fibroblastic migratory morphology or become incorporated in the forming bone tissue as osteocytes.

Using the same mouse model of Hic1Cre^{ERT2}/tdTomato, Soliman et al. have shown that Hic1⁺ cells are a heterogenous population of progenitor cells having two main subclusters of PDGFRa⁺/Sca1⁺ and PDGFRa⁺/Sca1⁻. The PDGFRa⁺/Sca-1⁺ subtype is a multipotent population of mesenchymal progenitors and upon injury some of the Sca1⁺ differentiate into Sca1⁻ subtype¹⁷. The anatomical locations of PDGFRa⁺/Sca-1⁺ and PDGFRa⁺/Sca-1⁻ cells were not clearly addressed in the myocardium by Soliman et al. However, in the cranium, there are three obvious sources of cells: the periosteum, the diplöe, and the dura mater also known as the endocranial periosteum. In our study, labeling was induced at post-natal week 8 following tamoxifen treatment, immunolabeling of tdtomato⁺ cells at week 14 post-labeling showed the presence of these cells in the periosteum, the outer layer of dura mater, and a few labeled cells in the diplöe. It should be noted that by 14 weeks the diplöe is a protected environment since the inner and outer tables of the cranium have closed at the osteotomy site (FIG. 4a) thus the diplöe can play no role in healing at such later time points (we did not prepare samples for immunohistochemistry at earlier time points). Therefore, we might have neglected the possible contribution of the diploe-derived-MPs. In response to implantation, we observed that few cells appeared to stem from the dura, and the majority of the tdTomato MPs resided in the inner layer of the ecto-cranial periosteum.

Together, these observations suggest that tissue-resident mesenchymal progenitor cells are localized between capillaries and enter the wound site where regeneration is needed along with capillary growth. However, this proliferating population of tissue resident progenitor cells are not pericytes, as initially thought to be in multiple organs including skeletal muscle. In fact, several studies have identified pericytes as tissue-resident progenitor cells in multiple human organs¹⁸⁻²⁰ by their expression of CD146, NG2, and PDGFRβ, and absence of hematopoietic, endothelial, and myogenic cell markers. Recently Guimarães-Camboa et al. (2017) challenged this premise by discovery of a novel gene, Tbx18, exclusively expressed by mural cells of adult organs. Their study indicates that PDGFRβ is not a reliable marker for pericyte lineage tracing²¹. A lineage tracing study using a Tbx18-CreERT2 mouse line revealed that despite obtaining promising in vitro data, pericytes and vascular smooth muscle cells (VSMCs) did not display endogenous multi-lineage potential during aging and injury. However, Guimarães-Camboa's findings are exclusive to mural cells and do not exclude the possibility of other cells existing in the perivascular niche that might have progenitor properties²¹. The findings of their study are aligned with our in vivo microscopical observations. However, they are in contradiction to what has been described by Diaz-Flores²², who contends that pericytes, specialized cells sharing the basement membrane with endothelial cells, are activated and separated from walls of the blood vessels to become transitional cells and ultimately differentiate into osteoblasts.

The divergence of these results helps distinguish between pericytes—cells sharing a basement membrane with endothelial cells—and perivascular cells, the cells that reside in the vicinity of the blood vessels. Specifically, the appearance of morphological distinct pericytes at later time points, and their absence in the blooming tDTomato population at earlier tiome points in our own work, would indicate that the progenitor population is not pericytic. However, it is clear that mural and perivascular cells of different organs are heterogenous populations, and thus the multipotency of these cells²³ at various developmental stages, or adult healing conditions, should be explored in their native environment.

Interestingly, our model shows that a massive proliferating bloom of MPs was observed in the recipients of the topographically complex implant, and the total number of tdTomato MPs was significantly higher compared to the smoother machined implant. This observation strengthens the notion that implant surface is the regulator of the extent of regeneration. The driving mechanism may be explained, in part, by our trajectory plots of individual cells in vitro, which showed that exposure to a local PL gradient not only provided stimulus for migration and recruitment of both endothelial and perivascular cells, but also controlled the directionality of migration for both cell types. Thus, during peri-implant healing the formation, and direction, of blood vessels⁵, is spatiotemporally associated with the ingress of mesenchymal progenitors to the wound site.

### Materials and Methods

### Animal Studies:

All animal procedures conducted in accordance with institutional animal use guidelines approved by University Health Network animal care committee (AUP #4884.1-2), Toronto, Ontario Canada.

### Mice

Hic1^{CreERT2} mice were sourced from the laboratory of one of us (TMU) at the University of British Columbia, Canada. For lineage tracing purpose, Hic1^{CreERT2} mice were interbred in-house with Rosa^{LSL-tdTomato} mice (The Jackson Laboratories stock #007914) to generate a mouse colony expressing tdTomato HiC1⁺ mesenchymal progenitor cells (MPs). To induce CRE-ERT2 nuclear translocation, 8 weeks-old mice were administered 100 μL per day intraperitoneally with 30 mg/mL of Tamoxifen in sunflower oil for 5 consecutive days. A 10-days washout period was considered before the mice were ready for experiments.

### Intravital Laser-Scanning Confocal Imaging:

Cranial implant window chamber placement surgeries were performed on mice and Intravital images were acquired using an LSM 710 (Zeiss, Germany) according to a previously described protocol¹⁴. Prior to each imaging session, mice were anesthetized by intraperitoneal injection of a mixture of Ketamine and Xylazine. Each mouse was administered 200 μm FITC-DEX (2 MDa; 0.1 mg/mouse) via the tail vein to visualize microvasculature (488 nm excitation, 500-550 nm emission). The imaging procedure was followed according to the experimental timeline shown in FIG. T1b. The images were acquired using 5×, 10×, and 20× water immersion objectives from the entire defect area including the implant as well as each of 4 healing volumes around the implant. Images were acquired at 1024×1024 pixels and 0.8 μs pixel dwell. tdTomato perivascular MPs were visualized in a second channel (561 nm excitation, 566-615 nm emission), the implant was visualized by collecting reflected light in a second channel (633 nm excitation, 622-666 nm emission). Two-photon laser scanning confocal microscopy was performed occasionally at late time points for label-free visualization of bone and bone matrix through second harmonic generation (SHG) (840 nm excitation, Emission 420 nm, 10% laser power). To obtain 3D images of the CIWC, stacks of images were collected in Z direction to size≅500 μm. Image acquisition settings were maintained consistently throughout all time points and groups.

### Image Processing and Analysis:

Image processing and analysis of the intravital confocal images was performed in Zen lite (Zeiss, Jena, Germany) and Imaris (ver. 8.3.0, Bitplane AG, Switzerland). Functional vessel density was obtained by calculating the positive pixel percentage of a z-stacks. Quantitative spatial analysis of cells in the in the peri-implant wound site has been performed using Imaris spots. The 4 healing volumes represented 4 ROIs that we identified and analyzed at each imaging time point.

### Ex-Vivo Tissue Histology

A 4 mm osteotomy was performed in the cranium of 10-week old Hic1/tdTomato mice. Implant-free window chambers were placed following the same protocol for CIWC placement. The mice were euthanized at day 42 post-implantation. The cranium was collected and assessed by ex-vivo histology for RFP (tdTomato), CD31 (blood vessels), as well as hematoxylin and eosin (H&E). Intact cranial bone collected from same age mice served as controls.

The animals were euthanized by exposure to CO₂ at days 43 post-surgery. After dissecting the skull, the mandible and the brain were removed. The samples were further trimmed to remove excess tissue and fixed in 4% Paraformaldehyde (PFA) for at 24 hrs. For histology, samples were decalcified using 14% EDTA solution in distilled water and embedded in paraffin. Coronal sections (6 μm) were obtained from the middle of the defect, consecutive slides were then stained for hematoxylin & eosin, Red fluorescent protein (RFP) (Abcam Cat. No. ab34771) for tdTomato cell, and CD31 (Abcam Cat. No. 28364) at 1:400 and 1:50 diluted in dako diluent (Dako Cat. No. S0809) for endothelial cells. Images were acquired using Aperio AT2 whole slide scanner (Leica, Canada) at 20× magnification.

### Cell Sources and Culture Conditions

Perivascular cells: human umbilical cord perivascular cells (HUCPVCs) isolated by physical extraction from umbilical cord vessels under a procedure performed by Tissue Regeneration Therapeutics Inc (Toronto, CA), followed by explant culture of perivascular tissue in serum-free conditions, passaged (at seeding density of 1,333.33 cells/cm²) and harvested at day 5 and 80% confluency at passage #3. TheraPEAK™ MSCGM-CD serum-free Mesenchymal Stem cell Growth Medium (Lonza; Cat. No. 00190632) medium was used for culture, which was changed every 3 days. TrypLE Select CTS (lnvitrogen; Cat. No. A1285901) was used for enzymatic dissociation at 80% confluency. HUCPVCs used for all in vitro migration and chemotaxis assays were pooled from 5 different donors.

Endothelial cells: human umbilical vein endothelial cells (HUVECs) were obtained from Tissue Regenerative Therapeutics Inc (Toronto, CA). Cells were cultured in Endothelial growth medium-2 (Lonza; CC-3162) supplemented with 2% serum and 1% Antibiotics at a seeding density of 0.1×10⁵ cells/cm². The medium was changed the day after seeding and every other day thereafter. Cells were pooled from 3 different donors and were harvested at passage 3 at 70-80% confluency for migration and chemotaxis experiments.

BM-MSCs: human BM-MSCs were provided by Tissue Regeneration Therapeutics Inc. Culture conditions and techniques were the same as described for HUCPVCs.

Flow cytometry: Briefly, 1×10⁵ frozen-thawed HUCPVCs were washed in PBS containing 1% BSA and 2 mM EDTA (flow buffer) and incubated for 30 minutes at 4° C. in the same buffer containing the following conjugated anti-human antibodies (at 1:5-1:20 dilutions): HLA-DR-FITC, CD31-FITC, CD45-FITC, CD10-FITC, CD142-APC and CD34-APC (eBioscience); CD90-FITC, CD73-PE, CD105-PE, CD166-PE, CD146-PE, CD140b-PE and MHC I-APC (BD Biosciences). The cell suspensions were then washed with flow buffer and resuspended in flow buffer. Immediately before analysis on the Cytomix FC 500 flow cytometer (Beckman Coulter), cells were stained with Propidium Iodide (PI) to exclude dead cells and 5000 live or PI-negative events were collected. Surface marker detection via antibodies was measured in FL1 for FITC, FL2 for PE and FL4 for APC. Flow cytometry data were analyzed using Kaluza Software (Beckman Coulter) and presented as a positive % expression or mean fluorescence intensity (MFI) which is a measure of the intensity of the signal.

Microarray: HUCPVCs and BM-MSCs were cultured on 6 well plates using the conditions previously described. The RNA was isolated when reached 80% confluency using Tri Reagent (Sigma) and later purified using RNeasy MinElute cleanup kit (Qiagen, Canada) as per manufacturer's instructions. RNA purity and yield were determined using the NanoDrop 1000 (Thermo Fisher Scientific, Wilmington, Del.), and quality with Agilent 2100 bioanalyzer (Agilent Technologies, Canada). 8 HUCPVC biological replicates and 7 BM-MSC biological replicates were used for microarray analysis using the GeneChip Human Gene 1.0 ST array (Affymetrix, Santa Clara, Calif.) as per manufacturer's instructions.

Real-time chemotaxis assay: chemotaxis of HUVECs and HUCPVCs towards various concentrations of the platelet lysate was analyzed in real-time using 2D chemotaxis u-slides (ibidi, Germany). Cells suspension in complete medium harvested at passage 3, were seeded in the observation area of the μ-slide according to the protocol provided by the supplier at a concentration of 1.5×10⁶ cells/ml. Chemotaxis μ-slide (ibidi GmbH) forms a diffusion-based gradient of soluble growth factors. Each setup is 10 mm wide and 200 um high. There are 3 setups on each slide with the size of a microscope slide. There are 3 channels in each setup. A narrow observation area connects two larger reservoirs. The cells are initially seeded in the observation area, the left reservoir is filled with chemoattractant and the right reservoir is filled with a culture medium. By diffusion, cells will be exposed by a linear gradient that will stay stable for 48 hours²⁴. The μ-slides were incubated at 37 C.° for 2 hours to allow enough time to the cells to adhere to the bottom of the device. The μ-slides has two reservoirs to either side of the observation area. The left reservoir was filled with 25, 50, 70% Platelet Lysate (PL) (Cook Medical, Bloomington, Ind.) in complete medium and the right reservoir with the complete medium. By diffusion, cells will be exposed to a linear gradient of a PL. Time-lapse video microscopy was conducted using inverted live cell microscope (Zeiss, Germany) with 4× objective for 24 hours to observe the chemotaxis of the cells in response to a linear gradient of platelet lysate. Cell tracking was performed with the manual tracking plugin in ImageJ. Chemotaxis and motility parameters, including forward migration index (FMIx, FMIy), mean velocity, and P-value of the Rayleigh test, were calculated and plotted using ibidi chemotaxis tools.

### Transwell Migration Assay:

The Boyden chamber migration assay was performed using Transwell inserts with 8 μm pores (Corning, N.Y.). A total of 50,000 in 200 μL cells were added to the top compartment of the inserts, which were placed into 12-well plates. The filters were transferred into wells containing 1000 μL 0, 10, 25, 50, 100% Platelet lysate (PL) in serum-free Lonza medium (SFM). The filters were collected from the well-plates after 12 and 6 hours of incubating at 37° C. for HUCPCs, and HUVECs respectively. Filters were fixed with 4% paraformaldehyde (PFA) and stained with Hoechst 33342 for nuclei. The number of cells that transmigrated to the underside of the filters were counted in each well using an inverted fluorescence microscope.

### Statistical Analysis

Temporal series results (Day 3 to 28) were presented as mean±SEM and analyzed by two-way repeated measures analysis of variance (ANOVA) in GraphPad. Bonferroni post-tests were performed to test the significance of the means between implant groups at each time point. A confidence level of 95% was considered significant. The intravital procedure was repeated with 6 to 8 animals per time point per implant group. P-values<0.01 were considered significant.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES 1

1. Davies, J. Understanding peri-implant endosseous healing. J. Dent. Educ. 67, 932-949 (2003).
2. Doherty, M. J. et al. Vascular pericytes express osteogenic potential in vitro and in vivo. J. Bone Miner. Res. 13, 828-838 (1998).
3. Crisan, M. et al. A Perivascular Origin for Mesenchymal Stem Cells in Multiple Human Organs. Cell Stem Cell 3, 301-313 (2008).
4. Bauer, S. M., Bauer, R. J, & Velazquez, O. C. Angiogenesis, Vasculogenesis, and Induction of Healing in Chronic Wounds. Vasc. Endovascular Surg. 39, 293-306 (2005).
5. Grant, M. B. et al. Adult hematopoietic stem cells provide functional hemangioblast activity during retinal neovascularization. Nat. Med. 8, 607-612 (2002).
6. Tonnesen, M. G., Feng, X. & Clark, R. A. F. Angiogenesis in wound healing. J. Investig. Dermatology Symp. Proc. 5, 40-46 (2000).
7. Clark, E. R. & Clark, E. L. Microscopic observations on the growth of blood capillaries in the living mammal. Am. J. Anat. 64, 251-301 (1939).
8. Djonov, V., Andres, A. C. & Ziemiecki, A. Vascular remodelling during the normal and malignant life cycle of the mammary gland. Microscopy Research and Technique 52, 182-189 (2001).
9. Patan, S. et al. Vascular morphogenesis and remodeling in a human tumor xenograft: blood vessel formation and growth after ovariectomy and tumor implantation. Circ. Res. 89, 732-739 (2001).
10. Kilarski, W. W., Sarnolov, B., Petersson, L., Kvanta, A. & Gerwins, P. Biomechanical regulation of blood vessel growth during tissue vascularization. Nat Med. 15, 657-664 (2009).
11. Styp-Rekowska, B., Hlushchuk, R., Pries, A. R. & Djonov, V. Intussusceptive angiogenesis: pillars against the blood flow. Acta physiologica (Oxford, England) 202, 213-223 (2011).
12. Buser, D. et al. Influence of surface characteristics on bone integration of titanium implants. A histomorphometric study in miniature pigs. J. Biomed. Mater. Res. 25, 889-902 (1991).
13. Shibli, J. A. et at. Influence of implant surface topography on early osseointegration: A histological study in human jaws. J. Biomed. Mater. Res.—Part B Appl. Biomater. 80, 377-385 (2007).
14. Mendes, V. C., Moineddin, R. & Davies, J. E. Discrete calcium phosphate nanocrystalline deposition enhances osteoconduction on titanium-based implant surfaces. J. Biomed. Mater. Res.—Part A 90, 577-585 (2009).
15. Nygren, H., Eriksson, C. & Lausmaa, J. Adhesion and activation of platelets and polymorphonuclear granulocyte cells at TiO2 surfaces. J. Lab. Clin. Med. 129, 35-46 (1997).
16. Park, J. Y., Gemmell, C. H. & Davies, J. E. Platelet interactions with titanium: Modulation of platelet activity by surface topography. Biomaterials 22, 2671-2682 (2001).
17. Campos, V. et al. Characterization of neutrophil adhesion to different titanium surfaces. Bull. Mater. Sci. 37, 157-166 (2014).
18. Kämmerer, P. W. et al. Early implant healing: Promotion of platelet activation and cytokine release by topographical, chemical and biomimetical titanium surface modifications in vitro. Clin. Oral Implants Res. 23, 504-510 (2012).
19. Refai, A. K., Textor, M., Brunette, D. M. & Waterfield, J. D. Effect of titanium surface topography on macrophage activation and secretion of proinflamrnatory cytokines and chemokines. J. Biomed. Mater. Res. A 70, 194-205 (2004).
20. Sainson, R. C. A. et al. TNF primes endothelial cells for angiogenic sprouting by inducing a tip cell phenotype. Blood 111, 4997-5007 (2008).
21. Peng, L. et al. Whole genome expression analysis reveals differential effects of TiO 2 nanotubes on vascular cells. Nano Lett. 10, 143-148 (2010).
22. Partida, E. B. et al. Improved in vitro angiogenic behavior on anodized titanium dioxide nanotubes. J. Nanobiotechnology 15, 1-21 (2017).
23. Donos, N. et al. Gene expression profile of osseointegration of a hydrophilic compared with a hydrophobic microrough implant surface. Clin. Oral Implants Res. 22, 365-372 (2011).
24. Chiappini, C. et al. Biodegradable silicon nanoneedles delivering nucleic acids intracellularly induce localized in vivo neovascularization. Nat. Mater. 14, 532-539 (2015).
25. Salou, L., Hoornaert, A., Louarn, G. & Layrolle, P. Enhanced osseointegration of titanium implants with nanostructured surfaces: An experimental study in rabbits. Acta Biomater. 11, 494-502 (2015).
26. Lausmaa, J., Kasemo, B. & Mattsson, H. Surface spectroscopic characterization of titanium implant materials. App. Surf. Sci. 44, 133-146 (1990).
27. Vincent, J. F. V. Applications—Influence of Biology on Engineering. J. Bionic Eng. 3, 161-177 (2006).
28. Bennett, R. E. et al. Tau induces blood vessel abnormalities and angiogenesis-related gene expression in P301L transgenic mice and human Alzheimer's disease. Proc. Natl. Acad. Sci. 115, E1289-E1298 (2018).
29. Maeda, A. et al. In vivo optical imaging of tumor and microvascular response to ionizing radiation. PLoS One 7, e42133 (2012).
30. Davies, J. E. Mechanisms of endosseous integration. Int. J. Prosthodont. 11, 391-401 (1998).
31. Liddell, R., Ajami, E. & Davies, J. Tau (τ): A New Parameter to Assess the Osseointegration Potential of an Implant Surface. Int. J. Oral Maxillofac. Implants 32, 102-112 (2017).
32. Huang, C, et al. Spatiotemporal Analyses of Osteogenesis and Angiogenesis via Intravital Imaging in Cranial Bone Defect Repair. J. Bone Miner. Res. 30, 1217-30 (2015).
33. Holstein, J. H. et al. Intravital microscopic studies of angiogenesis during bone defect healing in mice calvaria. Injury 42, 765-771 (2011).
34. Umoh, J. U. et al. In vivo micro-CT analysis of bone remodeling in a rat calvarial defect model. Phys. Med. Biol. 54, 2147-2161 (2009).
35. Kim, T.-H., Singh, R. K., Kang, M. S., Kim, J.-H. & Kim, H.-W. Gene delivery nanocarriers of bioactive glass with unique potential to load BMP2 plasmid DNA and to internalize into mesenchymal stem cells for osteogenesis and bone regeneration. Nanoscale 8, 8300-8311 (2016).
36. Ye. J. H. et al. Critical-size calvarial bone defects healing in a mouse model with silk scaffolds and SATB2-modified iPSCs. Biomaterials 32, 5065-5076 (2011).
37. Lienemann, P. S. at al. Longitudinal in vivo evaluation of bone regeneration by combined measurement of multi-pinhole SPECT and micro-CT for tissue engineering. Sci. Rep. 5, 10238 (2015).
38. Lo, S. C. et al. Enhanced critical-size calvarial bone healing by ASCs engineered with Cre/loxP-based hybrid baculovirus. Biomaterials 124, 1-11 (2017).
39. Davies, J. E., Mendes, V. C., Ko, J. C. H. & Ajami, E. Topographic scale-range synergy at the functional bone/implant interface. Biomaterials 35, 25-35 (2014).
40. Pham, J. T., Xue, L., Del Campo, A. & Salierno, M. Guiding cell migration with microscale stiffness patterns and undulated surfaces. Acta Biomater. 38, 106-115 (2016).
41. Uttayarat, P. et al. Microtopography and flow modulate the direction of endothelial cell migration. AJP Hear. Circ. Physiol. 294, H1027-H1035 (2008).
42. Culver, J. C., Vadakkan, T. J. & Dickinson, M. E. A Specialized Microvascular Domain in the Mouse Neural Stem Cell Niche. PLoS One 8, e53546 (2013).
43. Walchli, T. et al. Quantitative assessment of angiogenesis, perfused blood vessels and endothelial tip cells in the postnatal mouse brain. Nat. Protoc. 10, 53-74 (2014).
44. McKenzie, J. A. G. et al. Apelin is required for non-neovascular remodeling in the retina. Am. J. Pathol. 180, 399-409 (2012).
45. Seaman, M. E., Peirce, S. M. & Kelly, K. Rapid analysis of vessel elements (RAVE): A tool for studying Physiologic, Pathologic and Tumor Angiogenesis. PLoS One 6, e20807 (2011).
46. Cristofaro, B. et al. DII4-Notch signaling determines the formation of native arterial collateral networks and arterial function in mouse ischemia models. Development 140, 1720-9 (2013).
47. Kassab, G. S. Scaling laws of vascular trees: of form and function. AJP Hear. Circ. Physiol. 290, H894-H903 (2005).
48. Kochhan, E. et al. Blood Flow Changes Coincide with Cellular Rearrangements during Blood Vessel Pruning in Zebrafish Embryos, PLoS One 8, e75060 (2013).
49. Winkler, I. G. et al. Positioning of bone marrow hematopoietic and stromal cells relative to blood flow in vivo: Serially reconstituting hematopoietic stem cells reside in distinct nonperfused niches. Blood 116, 375-385 (2010).
50. Cleaver, O. & Krieg, P. A. in Heart Development and Regeneration 487-528 (Elsevier, 2010). doi:10.1016/B978-0-12-381332-9.00023-2
51. Colnot, C. Skeletal Cell Fate Decisions Within Periosteum and Bone Marrow During Bone Regeneration. J. Bone Miner. Res. 24, 274-282 (2009).
52. Ozerdem, O. R. et al. Roles of periosteum, dura, and adjacent bone on healing of cranial osteonecrosis. The Journal of craniofacial surgery 14, 371-372 (2003).
53. Levi, B. et al. Dura mater stimulates human adipose-derived stromal cells to undergo bone formation in mouse calvarial defects. Stem Cells 29, 1241-1255 (2011).
54. Crisan, M. et al, Perivascular Multipotent Progenitor Cells in Human Organs. Ann. N. Y. Acad. Sci. 1176, 118-123 (2009).
55. Guimaraes-Carnboa, N. et al. Pericytes of Multiple Organs Do Not Behave as Mesenchymal Stem Cells In Vivo. Cell Stem Cell 20, 345-359.e5 (2017).
56. Huang, C. et al. Spatiotemporal Analyses of Osteogenesis and Angiogenesis via Intravital Imaging in Cranial Bone Defect Repair. J. Bone Miner. Res. 30, 1217-30 (2015).
57. Ananth, H. et al. A Review on Biomaterials in Dental Implantology. Int. J. Biomed. Sci. 11, 113-20 (2015).
58. Planell, J. A. Biomaterials in orthopaedics. J. Chem. Technol. Biotechnol 5, 1137-58 (2008).
59. Mantripragada, V. P., Lecka-Czernik, B., Ebraheim, N. A, & Jayasuriya, A. C. An overview of recent advances in designing orthopedic and craniofacial implants. Journal of Biomedical Materials Research—Part A 101, 3349-3364 (2013).
60. Koshy, E., and Philip, S. R. Dental Implant Surfaces: An Overview. International Journal of Clinical Implant Dentristry, January-April 2015; 1(1): 14-22.
61. Smeets, R. et al. Impact of Dental Implant Surface Modifications on Osseointegration. BioMed Research International, Volumn 2016, Article ID 6285620, 16 pages.
62. Stanford, C. M. Surface Modification of Biomedical and Dental Implants and the Processes of Inflammation, Wound Healing and Bone Formation. Int. J. Mol. Sci. 2010, 11, 354-369.

### REFERENCES 2

1. Eming, S. A., Martin, P. & Tomic-Canic, M. Wound repair and regeneration: mechanisms, signaling, and translation. Sci. Transl. Med. 6, 265sr6 (2014).
2. Nygren, H., Eriksson, C. & Lausmaa, J. Adhesion and activation of platelets and polymorphonuclear granulocyte cells at TiO2 surfaces. J. Lab. Clin. Med. 129, 35-46 (1997).
3. Jetten, N. et al. Anti-inflammatory M2, but not pro-inflammatory M1 macrophages promote angiogenesis in vivo. Angiogenesis 17, 109-118 (2014).
4. Khosravi, N. et al. Intravital imaginal for tracking of angiogenesis and cellular events around surgical bone implants. Tissue Eng. Part C Methods ten.TEC.2018.0252 (2018). doi:10.1089/ten.TEC.2018.0252
5. Khosravi, N., Maeda, A., Dacosta, R. S. & Davies, J. E. Nanosurfaces modulate the mechanism of peri-implant endosseous healing by regulating neovascular morphogenesis. Commun. Biol. 1, 72 (2018).
6. Togarrati, P. P. et al. Identification and characterization of a rich population of CD34+ mesenchymal stern/stromal cells in human parotid, sublingual and submandibular glands. Sci. Rep. 7, 3484 (2017).
7. Carter, M. G. et al. Mice deficient in the candidate tumor suppressor gene Hic1 exhibit developmental defects of structures affected in the Miller-Dieker syndrome. Hum Mol Genet 9, 413-9 (2000).
8. Schreiner, P. Analysis of Hic1-expressing cells in the murine pancreas and their role in tissue regulation and regeneration. (2014). doi:10.14288/1.0167026
9. Uezumi, A. et al. Identification and characterization of PDGFR+ mesenchymal progenitors in human skeletal muscle. Cell Death Dis. 5, e1186-e1186 (2014).
10. Farahani, R. M. & Xaymardan, M. Platelet-derived growth factor receptor alpha as a marker of mesenchymal stem cells in development and stem cell biology. Stem Cells International 2015, 362753 (2015).
11. Paylor, B, Fernandes, J., McManus, B. & Rossi, F. Tissue-resident Sca1+ PDGFRα+ mesenchymal progenitors are the cellular source of fibrofatty infiltration in arrhythmogenic cardiomyopathy. F1000Research (2013). doi:10.12688/f1000research.2-141.v1
12. Carr, M. J. et al. Mesenchymal Precursor Cells in Adult Nerves Contribute to Mammalian Tissue Repair and Regeneration. Cell Stem Cell (2018). doi:10.1016/j.stem.2018.10.024
13. Park, J. Y., Gemmell, C. H. & Davies, J. E. Platelet interactions with titanium: Modulation of platelet activity by surface topography. Biomaterials 22, 2671-2682 (2001).
14. Khosravi, N. et al. Intravital imaging for tracking of angiogenesis and cellular events around surgical bone implants. Tissue Eng. Part C Methods ten.TEC.2018.0252 (2018). doi:10.1089/ten.TEC.2018.0252
15. Seynhaeve, A. L. B. et al. Spatiotemporal endothelial cell-pericyte association in tumors as shown by high resolution 4D intravital imaging. Sci. Rep. 8, 9596 (2018).
16. Sarugaser, R., Ennis, J., Stanford, W. L. & Davies, J. E. Isolation, propagation, and characterization of Human Umbilical Cord Perivascular Cells (HUCPVCs). Methods Mol. Biol. 482, 269-279 (2009).
17. Soliman, H. et al. Pathogenic potential of Hid expressing cardiac stromal progenitors. bioRxiv 544403 (2019). doi:10.1101/544403
18. Crisan, M. et al. A Perivascular Origin for Mesenchymal Stem Cells in Multiple Human Organs. Cell Stem Cell 3, 301-313 (2008).
19. Göritz, C, et al. A pericyte origin of spinal cord scar tissue. Science (80-.). 333, 238-242 (2011).
20. Dore-Duffy, P., Katychev, A., Wang, X. & Van Buren, E. CNS Microvascular Pericytes Exhibit Multipotential Stem Cell Activity. J. Cereb. Blood Flow Metab. 26, 613-624 (2006).
21. Guimaraes-Camboa, N. et al. Pericytes of Multiple Organs Do Not Behave as Mesenchymal Stem Cells In Vivo. Cell Stem Cell 1-15 (2017).
22. Diaz-Flores, L., Gutierrez, R., Lopez-Alonso, A., Gonzalez, R. & Varela, H. Pericytes as a supplementary source of osteoblasts in periosteal osteogenesis, Clin. Orthop. Relat. Res. 280-286 (1992).
23. Sarugaser, R., Hanoun, L., Keating, A., Stanford, W. L. & Davies, J. E. Human mesenchymal stem cells self-renew and differentiate according to a deterministic hierarchy. PLoS One 4, e6498 (2009).
24. Zengel, P. et al. μ-Slide Chemotaxis: A new chamber for long-term chemotaxis studies. BMC Cell Biol. 12, (2011).

1. A composition for promotion of wound healing, the composition comprising a micro- or nano-topographical complex surface.

2. The composition of claim 1, for modifying the rate, extent, location and directionality of neovascularization.

3. A product or device comprising the composition of claim 1.

4. The composition of claim 1, further comprising a biological component.

5. The composition of claim 1, further comprising a contrast agent.

6. (canceled)

7. (canceled)

8. The product or device of claim 3, wherein the product or device is a medical implant.

9. The composition of claim 1, comprising a metal piece having the micro- or nano-topographical complex surface.

10. The composition of claim 1, wherein the micro- or nano- topographical complex surface is comprised of microtubules, threading, pores, porous sinters, and/or microtextures.

11. The composition of claim 4, wherein the biological component comprises tissues, cells, exosomes, extracellular vesicles, microparticles, cytokines, antibiotics, antifungal drugs, anti-inflammatory drugs, nanoparticles, or media.

12. The composition of claim 5, wherein the contrast agent comprises fluorescent dyes, chromogenic dyes, quantum dots (QDots), Raman-active agents, molecular beacons, nanoparticles having fluorescent agents, scattering or absorbing nanoparticles, or biologically-activated/sensitive contrast agents.

13. The product or device of claim 3, wherein the product or device is a skin dressing, bandage, scaffold, patch, implant, thin film, wire, catheter, mesh, nanowire, or implantable vascular beds.
